(19) United States
(12) Patent Application Publication
(10) Pub. No.: US 2010/0022541 A1
(43) Pub. Date: Jan. 28, 2010

(54) Chemical inhibitors of bacterial heptose synthesis, methods for their preparation and biological applications of said inhibitors

(76) Inventors: Sonia Escaich, Paris (FR); Alexis Denis, Paris (FR); Francois Moreau, Orsay (FR); Vincent Gerusz, Paris (FR); Nicolas Desroy, Sceaux (FR)

Correspondence Address: NIXON & VANDERHYE, PC, 901 NORTH GLEBE ROAD, 11TH FLOOR, ARLINGTON, VA, 22203, US

(21) Appl. No.: 12/311,278

(22) PCT Filed: Sep. 25, 2007
(86) PCT No.: PCT/IB2007/003276

§ 371 Date: Mar. 25, 2009

Related U.S. Application Data
(60) Provisional application No. 60/846,735, filed on Sep. 25, 2006.

(51) Int. Cl. A61K 31/5377; C12Q 1/66; C07D 413/12; A61K 31/437

(52) U.S. Cl. 514/236.8; 435/8; 546/271.4; 514/340; 544/124

(57) ABSTRACT

The invention relates to new compounds having heptose synthesis inhibitory properties, of formula (I) or a pharmaceutically acceptable salt, or prodrug thereof, wherein A is an aryl or heterocycle, optionally substituted by one or several identical or different R such as H, C1-C10 alkyl, C1-C10 alkyl-OR₁, C1-C10 alkyl-NR₁R₁, alkoxy, hydroxy, thioalkyl, aryl, heterocycle, halogen, nitro, cyano, CO₂R₁, NR₁R₁, NR₁C(O)R₁, C(O)NR₁R₁, NR₁C(S)R₁, C(S)NR₁R₁, SO₂NR₁R₁, SO₂R₁, NR₁SO₂R₁, NR₁C(O)NR₁R₁, NR₁C(O)OR₁, NR₁C(S)NR₁R₁, NR₁C(S)OR₁, R₁C═NOR₁, C(O)R₁, aryloxy, thioaryl, alkenyl, alkynyl R1 identical or different is H or C1-C10 alkyl B₁, B₂, B₃ identical or not represent C, N, O, S to form a five-membered aromatic ring wherein from one to three carbon atoms are replaced by a heteroatom selected from S, O, N optionally substituted by one or several identical or different R such as defined above B₄ is C or N Y is H, C1-C10 alkyl, alkoxy, thio-alkyl, optionally substituted by one or several identical or different R such as defined above W is C, O or N, substituted or not by one or several C1-C10 alkyl radicals D is an heterocycle optionally substituted by one or several identical or different R such as defined above.

The invention relates to new compounds capable of inhibiting bacterial heptose synthesis.

It also relates to their synthesis and the biological applications of the inhibitors for preventing or treating bacterial infections.

The lipopolysaccharide is a major component of the outer membrane of gram-negative bacteria. It is composed of three regions: the lipid A, the core oligosaccharide and the O antigen. The core oligosaccharide is divided into the inner core and the outer core. The inner core consists in a motif of five sugars: two Kdo (Kdo: 3-deoxy-D-manno-octulosonic acid) and three successive heptoses.

The first heptose transfer is catalysed by the Heptosyltransferase I (protein waaC) and the second heptose transfer by the Heptosyltransferase II (protein waaF).

The natural donor substrate of these transferases is ADP heptose, which is synthesized in bacteria from sedoheptulose by the successive enzymatic steps catalyzed by the following enzymes: GmhA, RfaE, GmhB, and RfaD (WaaD) (Journal of Bacteriology, January 2002, p 363-369).

Heptose synthetic pathway is conserved among gram negative bacterial species and is necessary for full LPS synthesis. It has been demonstrated that a complete LPS is necessary for pathogenesis due to the gram negative bacteria. Bacteria lacking heptoses do have a rough phenotype because of the absence of the carbohydrate chains of the inner and outer core LPS. Bacteria having this phenotype are unable to give a productive infection in the host and in particular are very sensitive to the bactericidal effect of complement.

Compounds inhibiting heptose synthesis activity are expected to prevent full LPS synthesis in gram negative bacteria, inducing a high sensitivity to the complement and inhibiting bacterial multiplication in the blood.

Therefore small molecules inhibitors of heptose synthesis could be a new way to treat bloodstream infections by pathogenic bacteria.

It is known that the reactions catalyzed by RfaE are essential for heptose synthesis. As shown in WO 2006/058 796, this enzyme is essential for pathogenicity in an experimental model of infection.

To search for inhibitors of this enzyme, a new biochemical assay has been established by the inventors. They have also elaborated synthesis protocols to obtain the new inhibitors.

Accordingly, it is an object of the invention to provide new inhibitors of bacterial heptose synthesis to by inhibiting the gene product of RfaE which is necessary for the pathogenicity of Gram-negative bacteria responsible for severe infections such as the Gram negative species (spp.): Escherichia coli, Enterobacter, Salmonella, Shigella, Pseudomonas, Acinetobacter, Neisseria, Klebsiella, Serratia, Citrobacter, Proteus, Yersinia, Haemophilus, Legionella, Moraxella and Helicobacter pylori.

Another object is to provide methods for preparing such inhibitors by chemical synthesis.

Still another object of the invention is to provide new drugs, methods of prevention and therapeutical treatment of severe infections due to gram negative bacteria.

Still another object of the invention is to provide drugs containing in their active principle at least one of said inhibitory molecules or one of said inhibitory molecules in combination with an antimicrobial peptide or a natural, hemisynthetic or synthetic antibacterial molecule.

This is also an aim of the invention to provide a method for assessing the inhibitory properties of said inhibitors.

The present invention relates then to compounds of formula I:

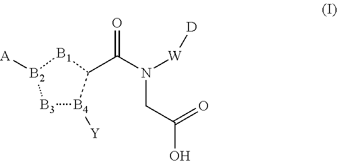

or a pharmaceutically acceptable salt or prodrug thereof, wherein

A is an aryl or heterocycle, optionally substituted by one or several identical or different R such as H, C1-C10 alkyl, C1-C10 alkyl-OR₁, C1-C10 alkyl-NR₁R₁, alkoxy, hydroxy, thioalkyl, aryl, heterocycle, halogen, nitro, cyano, CO₂R₁, NR₁R₁, NR₁C(O)R₁, C(O)NR₁R₁, NR₁C(S)R₁, C(S)NR₁R₁, SO₂NR₁R₁, SO₂R₁, NR₁SO₂R₁, NR₁C(O)NR₁R₁, NR₁C(O)OR₁, NR₁C(S)NR₁R₁, NR₁C(S)OR₁, R₁C═NOR₁, C(O)R₁, aryloxy, thioaryl, alkenyl, alkynyl

R1 identical or different is H or C1-C10 alkyl

B₁, B₂, B₃ identical or not represent C, N, O, S to form a five-membered aromatic ring wherein from one to three carbon atoms are replaced by a heteroatom selected from S, O, N optionally substituted by one or several identical or different R such as defined above

B₄ is C or N

Y is H, C1-C10 alkyl, alkoxy, thioalkyl, optionally substituted by one or several identical or different R such as defined above

W is C, O or N, substituted or not by one or several C1-C10 alkyl radicals

D is an heterocycle optionally substituted by one or several identical or different R such as defined above

In a preferred embodiment, the present invention provides a compound of formula I or a pharmaceutically acceptable salt, or prodrug thereof, wherein

A is an aryl or an heterocycle optionally substituted by one or several identical or different R such as defined above

B₁, B₂, B₃, identical or not represent C, N, O, S to form a five-membered aromatic ring wherein from one to three carbon atoms are replaced by a heteroatom selected from S, O, N substituted or not by a C1-C10 alkyl

B₄ is C or N

Y is H or C1-C10 alkyl optionally substituted by one or several identical or different R such as defined above

W is C substituted or not by one or several C1-C10 alkyl radicals

D is a thiazole, benzothiazole, pyridine, or quinoline optionally substituted by one or several identical or different R such as defined above.

In another preferred embodiment, the invention relates to derivatives wherein A is an aryl optionally substituted by one or several identical or different R such as above defined.

Advantageously, A is an heterocycle optionally substituted by one or several identical or different R such as defined above.

In preferred derivatives, Y is a methyl or trifluoromethyl.

In more preferred derivatives, D is a 2-thiazole, 2-benzothiazole, 2-pyridine, or 2-quinoline optionally substituted by one pr several identical or different R such as defined above.

The meaning of any substituent R at any one occurrence is independent of its meaning, or any other substituents' meaning, at any other occurrence.

“C1-C10 alkyl” as applied herein means linear, branched or cyclic hydrocarbon groups having 1 to 10 carbon atoms preferably methyl, ethyl, n-propyl, isopropyl, n-butyl, isobutyl and t-butyl, pentyl, n-pentyl, isopentyl, neopentyl, hexyl, octyl, cyclopropyl cyclobutyl, cyclopentyl, cyclohexyl;

Alkoxy and thioalkyl mean any O or S atom substituted by a substituted or not C1-C10 alkyl group. Aryloxy, thioaryl, N-aryl, mean any O, S, N substituted by a substituted or not aryl, or heterocyclic group.

Ar or aryl means optionally substituted phenyl, naphtyl groups. Alkenyl and alkynyl mean optionally substituted C═C or C≡C groups.

Halogen or halo means F, Cl, Br, and I.

Het or heterocycle, indicates an optionally substituted five or six membered monocyclic ring, or a nine or ten-membered bicyclic ring containing one to five heteroatoms chosen from the group of nitrogen, oxygen and sulfur, which are stable and available by conventional chemical synthesis. Illustrative heterocycles are benzofuryl, benzimidazolyl, benzopyranyl, benzothienyl, furyl, imidazolyl, pyrazolyl, isoxazolyl, isothiazolyl, tetrazolyl, triazolyl, oxadiazolyl, indolinyl, morpholinyl, piperidinyl, piperazinyl, pyrrolyl, pyrrolidinyl, tetrahydropyridinyl, pyridinyl, thiazolyl, thienyl, benzothiazolyl, quinolinyl, isoquinolinyl, tetra- and perhydro-quinolinyl and isoquinolinyl, pyrazinyl, pyrazidinyl, triazinyl, purinyl, indolyl, indazolyl, pyrimidinyl, pyridonyl, oxazolyl, tetrahydropyranyl, tetrahydrofuranyl, [1,2,4]triazolo[1,5-a]pyridinyl, thiazolopyridinyl, thiazolopyrimidinyl, thiazolopyrazinyl, tetrahydrobenzothiazolyl.

Any C1-C10 alkyl, heterocycle, aryl, alkoxy, thioalkyl, aryloxy, thioaryl, N-aryl, alkenyl, alkynyl may be optionally substituted with the R group such as defined above or a non exclusive combination of different R values, which may be on any atom that results in a stable structure and is available by conventional synthetic techniques.

Also included in this invention are pharmaceutically acceptable organic or mineral salts of the compounds of this invention.

Also included in this invention are prodrugs of the compounds of this invention. Prodrugs are considered to be any covalently bonded carriers which release the active parent drug according to formula (I) in vivo.

In cases wherein the compounds of this invention may have one or more chiral centers, unless specified, this invention includes each unique racemic compound, as well as each unique nonracemic mixture.

In cases in which compounds have unsaturated carbon-carbon double bonds, both the cis (Z) and trans (E) isomers are within the scope of this invention.

In cases wherein compounds may exist in tautomeric forms, such as keto-enol tautomers, both forms are being included within this invention, whether existing in equilibrium or locked in one form by appropriate substitution.

Compounds of formula I and salts of such compounds having at least one salt forming group, as well as other components as thereafter defined may be prepared by any processes known to be applicable to the preparation of chemically related compounds. Such processes may use known starting materials or intermediates which may be obtained by standard procedures of organic chemistry. The following processes provide a variety of non-limiting routes for the production of the compounds of formula I and their intermediates. These processes constitute further features of the present invention.

The invention also relates to a process for preparing the above defined compounds.

Compounds of formula I and salts thereof may then be prepared by reaction of compounds of formula II or a salt thereof:

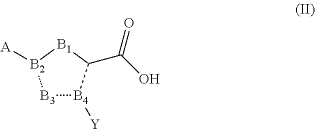

wherein A, B₁, B₂, B₃, B₄ and Y are as above defined; with a compound of formula III or a salt thereof:

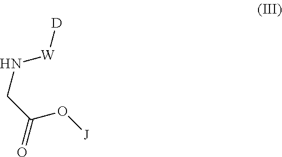

wherein D and W are as above defined, J is a C1-C10 alkyl group optionally substituted by one or several identical or different R such as defined above.

Formation of the amide bond can be achieved using a variety of known methods to activate the carboxylic acid functionality (non-limiting examples are peptide coupling reagents or formation of the acyl chloride). Conversion of the ester into the corresponding carboxylic acid can be achieved by hydrolysis, saponification, or any common deprotection reaction well known to those of ordinary skill in the art.

Alternatively, compounds of formula I and salts thereof may be prepared by reaction of compounds of formula IV, or a salt thereof:

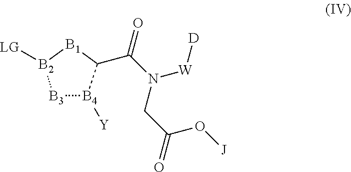

wherein B₁, B₂, B₃, B₄, D, W and Y are as above defined, LG is a leaving group such as a halogen or a sulfonyloxy group (non-limiting examples are chlorine, mesylate, triflate), J is a C1-C10 alkyl group optionally substituted by one or several identical or different R such as defined above; with a compound of formula V, or a salt thereof:

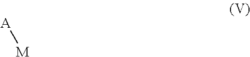

wherein A is as above defined, M represents H, B(OH)₂, B(OR)₂, BF₃K, or any metal atom substituted or not by R groups different or not, with R as above defined. Displacement of the leaving group of IV occurs by nucleophilic substitution or metal-mediated coupling reaction. Conversion of the ester into the corresponding carboxylic acid can be achieved by hydrolysis, saponification, or any common deprotection reaction well known to those of ordinary skill in the art.

Compounds of formula I and salts thereof may also be prepared by reaction of compounds of formula VI, or a salt thereof:

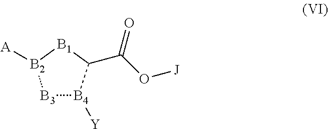

wherein A, B₁, B₂, B₃, B₄, Y are as above defined, J is a C1-C10 alkyl group optionally substituted by one or several identical or different R such as defined above; with a compound of formula III, or a salt thereof as above described. Formation of the amide bond can be achieved using a variety of known amidification procedures. Conversion of the ester into the corresponding carboxylic acid can be achieved by hydrolysis, saponification, or any common deprotection reaction well known to those of ordinary skill in the art.

The compounds of formula I and salts thereof thus obtained might undergo further transformations (such as deprotection, alkylation, acylation, nucleophilic substitution, reduction, oxidation, transition metal catalyzed reaction) to provide other compounds of formula I and salts thereof.

Compounds of formula II and salts thereof are known starting materials or intermediates which may be obtained by standard procedures of organic chemistry. Compounds of formula II can be obtained by saponification or hydrolysis of an ester, or by any other common deprotection reaction of protected acid functionalities of compounds of formula VI or a salt thereof as described herein before.

Compounds of formula VI and salts thereof can be synthesized by reaction of compounds of formula VII or a salt thereof:

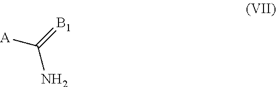

wherein A is as above defined and B₁ is O or S; with a compound of formula VIII or a salt thereof:

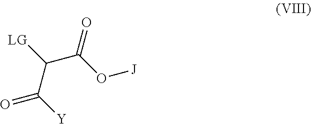

wherein Y is as above defined, LG is a leaving group such as a halogen or a sulfonyloxy group (non-limiting examples are chlorine, mesylate, triflate), J is a C1-C10 alkyl group optionally substituted by one or several identical or different R such as defined above. The reaction conditions for this process are well described in the literature (see for example: Bioorg. Med. Chem. Lett. 2003, 13, 1517).

Alternatively, compounds of formula VI and salts thereof can be synthesized by reaction of compounds of formula IX, or a salt thereof:

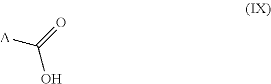

wherein A is as above defined; with a compound of formula X or a salt thereof:

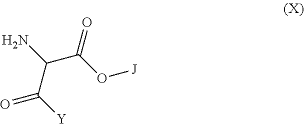

wherein Y is as above defined, J is a C1-C10 alkyl group optionally substituted by one or several identical or different R such as defined above. Such a procedure to synthesize oxazole rings is well described in the literature (see for example: Eur. J. Med. Chem.—Chimica Therapeutica 1976, 11, 263).

Compounds of formula VI, and salts thereof can also be prepared by the reaction of compounds of formula VII or a salt thereof as above defined, with a compound of formula XI or a salt thereof:

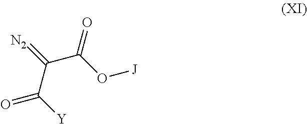

wherein Y is as above defined, J is a C1-C10 alkyl group optionally substituted by one or several identical or different R such as defined above. Such reaction conditions to obtain 5-membered heterocycles are well described in the literature (see for example: Tetrahedron 2004, 60, 3967).

Compounds of formula VI and salts thereof can also be prepared by the reaction of compounds of formula XII or a salt thereof:

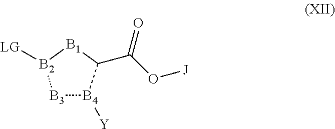

wherein B₁, B₂, B₃, B₄, and Y are as above defined; LG is a leaving group such as a halogen or a sulfonyloxy group (non-limiting examples are chlorine, mesylate, triflate), J is a C1-C10 alkyl group optionally substituted by one or several identical or different R such as defined above; with a compound of formula V, or a salt thereof as above described. Compounds of formula XII and salts thereof are known starting materials or intermediates which may be obtained by standard procedures of organic chemistry. Displacement of the leaving group of XII occurs by nucleophilic substitution or metal-mediated coupling reaction, such processes are described in the literature (see for example: Org. Lett. 2002, 4, 1363 and Tetrahedron Lett. 2004, 45, 3797).

The compounds of formula VI and salts thereof thus obtained might undergo further transformations (such as deprotection, alkylation, acylation, nucleophilic substitution, reduction, oxidation, transition metal catalyzed reaction) well known to those of ordinary skill in the art to provide other compounds of formula VI and salts thereof.

Compounds of formula II and salts thereof can also be prepared by reaction of a compound of formula XIII or a salt thereof:

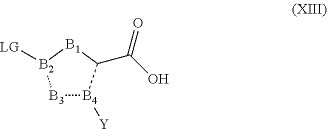

wherein B₁, B₂, B₃, B₄ and Y are as above defined, LG is a leaving group such as a halogen or a sulfonyloxy group (non-limiting examples are chlorine, mesylate, triflate); with a compound of formula V, or a salt thereof as above defined by nucleophilic substitution or metal-mediated coupling reaction, such process is described in the literature (see for example: J. Org. Chem. 2003, 68, 4302). Compounds of formula XIII and salts thereof are known starting materials or intermediates which may be obtained by standard procedures of organic chemistry.

The compounds of formula II and salts thereof thus obtained might undergo further transformations (such as deprotection, alkylation, acylation, nucleophilic substitution, reduction, oxidation, transition metal catalyzed reaction) well known to those of ordinary skill in the art to provide other compounds of formula II and salts thereof.

Compounds of formula III and salts thereof may be prepared by reaction of a compound of formula XIV, or a salt thereof:

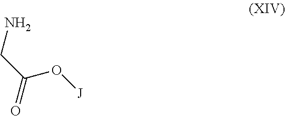

wherein J is a C1-C10 alkyl group optionally substituted by one or several identical or different R such as defined above; with a compound of formula XV, or a salt thereof:

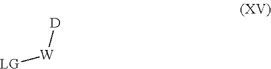

wherein D and W are as above defined and LG is a leaving group such as a halogen or a sulfonyloxy group (non-limiting examples are chlorine, mesylate, triflate). Such nucleophilic substitution is well described in the literature (see for example Heterocycles 1981, 1271).

Alternatively, compounds of formula III and salts thereof may be prepared by reaction of a compound of formula XVI, or a salt thereof:

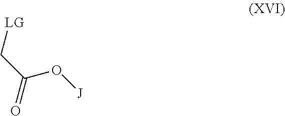

wherein LG is a leaving group such as a halogen or a sulfonyloxy group (non-limiting examples are chlorine, mesylate, triflate), J is a C1-C10 alkyl group optionally substituted by one or several identical or different R such as defined above; with a compound of formula XVII, or a salt thereof:

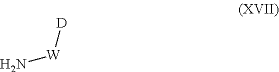

wherein D and W are as above defined. Such nucleophilic substitution is well described in the literature (see for example J. Chem. Soc. Perkin Trans. 1 1991, 2417).

Compounds of formula III and salts thereof can also be prepared by reaction of a compound of formula XVIII, or a salt thereof:

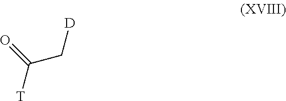

wherein D is as above defined and T is H or C1-C10 alkyl as defined herein previously; with a compound of formula XIV or a salt thereof as defined herein before. Such reductive amination procedure is well described in the literature (see for example Tetrahedron 2003, 50, 7103).

Compounds of formula III and salts thereof may also be synthesized by reaction of a compound of formula XIX, or a salt thereof:

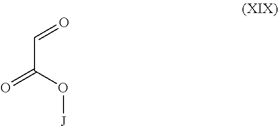

wherein J is a C1-C10 alkyl group optionally substituted by one or several identical or different R such as defined above; with a compound of formula XVII, or a salt thereof, as above defined. Such reductive amination procedure is well described in the literature (see for example J. Org. Chem. 1996, 61, 3849).

The compounds of formula III and salts thereof thus obtained might undergo further transformations (such as deprotection, alkylation, acylation, nucleophilic substitution, reduction, oxidation, transition metal catalyzed reaction) well known to those of ordinary skill in the art to provide other compounds of formula III and salts thereof.

Compounds of formula IV and salts thereof can be prepared by reaction of a compound of formula XIII or a salt thereof with a compound of formula III or a salt thereof, as defined herein previously. Formation of the amide bond can be achieved using a variety of known methods to activate the carboxylic acid functionality (non-limiting examples are peptide coupling reagents or formation of the acyl chloride).

Said chemical compounds are potent inhibitors of the enzymatic activity of RfaE as illustrated by the examples.

The invention thus also relates to a composition comprising at least a derivative of formula (I) such as above defined for use as drug.

It particularly relates to a composition for use as antibacterial agent against Gram-negative bacteria. Such a composition is particularly efficient to treat infections due to following Gram negative species (spp): Escherichia coli, Enterobacter, Salmonella, Shigella, Pseudomonas, Acinetobacter, Neisseria, Klebsiella, Serratia, Citrobacter, Proteus, Yersinia, Haemophilus, Legionella, Moraxella and Helicobacter pylori.

It also relates to a pharmaceutical composition comprising an effective amount of at least a derivative of formula (I) such as above defined, in combination with a pharmaceutically acceptable carrier.

Said pharmaceutical compositions are formulated to be administered for example under oral, injectable, parenteral routes, with individual doses appropriate for the patient to be treated.

The invention also relates to a method of treatment of microbial infections which comprises administering to a patient in need thereof an efficient amount of a pharmaceutical composition such as above defined.

According to another object, the invention also relates to a method for assessing RfaE enzymatic activity.
Said method comprises
pre-incubating at room temperature
DMSO or inhibitor to be tested dissolved in DMSO and RfaE in an assay buffer
and either
adding a reaction mixture composed of RfaE, β-heptose-7-phosphate, ATP, in the assay buffer and incubating at room temperature
adding a revelation mixture composed of luciferase, D-luciferin and N-acetylcysteamine
measuring the luminescence intensity and converting into inhibition % to further calculate the IC₅₀ values;
or
adding a reaction mixture composed of RfaE, β-heptose-7-phosphate ATP, pyruvate kinase, phosphoenolpyruvate, lactate dehydrogenase and NADH in said assay buffer,
measuring the fluorescence intensity of NADH kinetically and deriving inhibition % from fitted initial velocities, to further calculate the IC₅₀ values.

Other characteristics and advantages of the invention are given hereinafter.

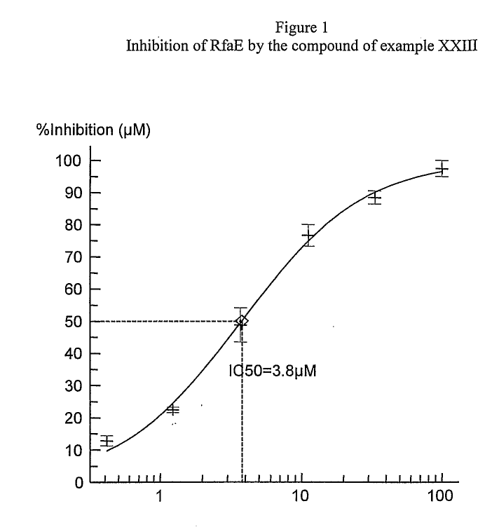

In the examples, it is referred to FIG. 1 which illustrates the dose dependent inhibition of RfaE biochemical activity by a compound according to the invention.

Proton nuclear magnetic resonance (¹H NMR) spectra were recorded on a 400 MHz Brüker instrument, and chemical shifts are reported in parts per million (6) downfield from the internal standard tetramethylsilane (TMS). Abbreviations for. NMR data are as follows: s=singlet, d=doublet, t=triplet, q=quadruplet, m=multiplet, dd=doublet of doublets, dt=doublet of triplets, br=broad. J indicates the NMR coupling constant measured in Hertz. CDCl₃ is deuteriochloroform, DMSO-d₆ is hexadeuteriodimethylsulfoxide, and CD₃OD is tetradeuteriomethanol. Mass spectra were obtained using electrospray (ES) ionization techniques on an Agilent 1100 Series LCMS. HPLC (analytical and preparative) were performed on an Agilent 1100 HPLC with DAD (Diode Array Detection). Preparative HPLC were performed at 0.7 mL/min on a Thermo Electron, Hypersil BDS C-18 column (250×4.6 mm, 5 μm) using a gradient of TFA 0.1% in water (50% to 100% and back to 50%) in ACN. Analtech Silica Gel GF and E. Merck Silica Gel 60 F-254 thin layer plates were used for preparative an analytical thin layer chromatography (TLC) respectively. Flash chromatography was carried out on Flashsmart Pack cartridge, irregular silica 40-60 μm or spherical silica 20-40 μm.

The meaning of certain abbreviations is given herein. ESI refers to electrospray ionization, HPLC refers to high pressure liquid chromatography, LCMS refers to liquid chromatography coupled with a mass spectrometer, M in the context of mass spectrometry refers to the molecular peak, MS refers to mass spectrometer, NMR refers to nuclear magnetic resonance, pH refers to potential of hydrogen, TFA refers to trifluoroacetic acid, DTT refers to dithiothreitol, TLC refers to thin layer chromatography.

The starting materials are commercially available unless indicated otherwise.

### EXAMPLE I

### {[[5-(benzyloxy)methyl-2-phenyl-1,3-oxazol-4-yl]carbonyl](pyridin-2-ylmethyl)amino}acetic Acid

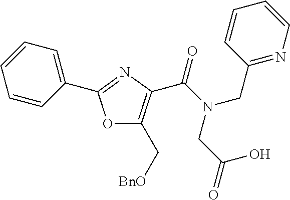

a)

A solution of 4-(acetylamino)benzenesulfonyl azide (1.77 g, 7.4 mmol) in anhydrous acetonitrile (30 mL) was stirred mechanically under argon at 0° C. A solution of ethyl 4-(benzyloxy)-3-oxobutanoate (1.45 g, 6.1 mmol, prepared as in Synthesis 1995, 1014) in acetonitrile (10 mL) was added, followed by triethylamine (2.6 mL, 18.7 mmol). The reaction mixture was stirred overnight allowing the temperature to rise to room temperature. The reaction mixture was filtered; the solid rinsed with diethyl ether and the filtrate was concentrated. The crude product was purified by flash chromatography (silica gel, cyclohexane/ethyl acetate 9/1) to afford ethyl 4-(benzyloxy)-2-diazo-3-oxobutanoate (1.45 g, 91%) as a bright yellow oil. ¹H NMR (CDCl₃), δ (ppm): 7.41-7.28 (m, 5H), 4.67 (s, 2H), 4.62 (s, 2H), 4.29 (q, J=7.2 Hz, 2H), 1.33 (t, J=7.2 Hz, 3H).

b)

Under argon, a solution of 4-(benzyloxy)-2-diazo-3-oxobutanoate (1.5 g, 5.5 mmol) in degassed 1,2-dichloroethane (11 mL) was slowly added (over a period of 2 hours) to a refluxing solution of benzamide (804 mg, 6.6 mmol) and rhodium (II) acetate dimer (61 mg, 0.14 mmol) in 1,2-dichloroethane (11 mL). The reaction was kept stirring under reflux overnight, then cooled to room temperature. An aqueous solution of ammonium chloride was added and the reaction mixture was extracted with dichloromethane. The combined organic extracts were dried over sodium sulfate, filtered and evaporated. The crude product was purified by flash chromatography (silica gel, cyclohexane/ethyl acetate 9/1 to 7/3) to afford ethyl 2-(benzoylamino)-4-(benzyloxy)-3-oxobutanoate (347 mg, 18%) as a yellow oil.

¹H NMR (CDCl₃), δ (ppm): 7.85 (d, J=7.2 Hz, 2H), 7.58-7.27 (m, 8H), 5.61 (d, J=7.2 Hz, 1H), 4.67 (s, 2H), 4.51 (d, J=7.6 Hz, 2H), 4.27 (q, J=7.2 Hz, 2H), 1.28 (t, J=7.2 Hz, 3H).

c)

A solution of ethyl 2-(benzoylamino)-4-(benzyloxy)-3-oxobutanoate (318 mg, 0.89 mmol) and phosphorus oxychloride (840 μL, 9 mmol) in anhydrous chloroform (9 mL) was stirred under argon at 90° C. overnight. The reaction mixture was cooled to 0° C., an aqueous solution of sodium bicarbonate was carefully added to quench the reaction media. The reaction mixture was extracted with dichloromethane. The combined organic extracts were dried over sodium sulfate, filtered and evaporated. The crude product was purified by flash chromatography (silica gel, cyclohexane/ethyl acetate 9/1 to 8/2) to afford ethyl 5-[(benzyloxy)methyl]-2-phenyl-1,3-oxazole-4-carboxylate (188 mg, 62%) as an orange solid.

¹H NMR (CDCl₃), δ (ppm): 8.15-8.13 (m, 2H), 7.51-7.48 (m, 3H), 7.40-7.28 (m, 5H), 4.99 (s, 2H), 4.67 (s, 2H), 4.44 (q, J=7.2 Hz, 2H), 1.41 (t, J=7.2 Hz, 3H).

d)

Lithium hydroxide (67 mg, 2.8 mmol) was added to a solution of ethyl 5-[(benzyloxy)methyl]-2-phenyl-1,3-oxazole-4-carboxylate (188 mg, 0.56 mmol) in tetrahydrofuran (4 mL) and water (4 mL). The reaction mixture was stirred at room temperature overnight. The solvents were removed under reduced pressure, then an aqueous hydrochloric solution was added and the reaction mixture was extracted with diethyl ether and ethyl acetate. The combined organic extracts were dried over sodium sulfate, filtered and evaporated. The crude product was purified by flash chromatography (silica gel, dichloromethane/methanol 98/2 to 95/5) to afford 5-[(benzyloxy)methyl]-2-phenyl-1,3-oxazole-4-carboxylic acid as a beige solid (158 mg, 91%).

ESI-MS m/z 310 (M+H)⁺.

### e) Representative Procedure for the Coupling of Carboxylic Acids and Secondary Amines:

A mixture of 5-[(benzyloxy)methyl]-2-phenyl-1,3-oxazole-4-carboxylic acid (40.5 mg, 0.13 mmol), N-(3-dimethylaminopropyl)-N′-ethylcarbodiimide hydrochloride (49.8 mg, 26 mmol), 4-dimethylaminopyridine (47.2 mg, 0.39 mmol) and methyl [(pyridin-2-ylmethyl)amino]acetate (28 mg, 0.16 mmol, prepared according to Bull. Chem. Soc. Jpn. 2002, 2423) in dichloromethane (2 mL) was stirred under argon at room temperature for 0.5 h and then at 50° C. overnight. An aqueous solution of ammonium chloride was added and the reaction mixture was extracted with dichloromethane. The combined organic extracts were dried over sodium sulfate, filtered and evaporated. The crude product was purified by preparative TLC (silica gel, dichloromethane/methanol 9/1) to afford methyl {[[5-(benzyloxy)methyl-2-phenyl-1,3-oxazol-4-yl]carbonyl](pyridin-2-ylmethyl)amino}acetate (47 mg, 77%).

ESI-MS m/z 472 (M+H)⁺.

### f) Representative Procedure for the Saponification of Esters:

A mixture of methyl {[[5-(benzyloxy)methyl-2-phenyl-1,3-oxazol-4-yl]carbonyl](pyridin-2-ylmethyl)amino}acetate (47 mg, 0.1 mmol) and lithium hydroxide (11.9 mg, 0.5 mmol) in tetrahydrofuran (1 mL) and water (1 mL) was stirred at room temperature overnight. The reaction mixture was then concentrated. The crude product was purified by preparative TLC (silica gel, dichloromethane/methanol 9/1) to afford {[[5-(benzyloxy)methyl-2-phenyl-1,3-oxazol-4-yl]carbonyl](pyridin-2-ylmethyl)amino}acetic acid (19 mg, 41%) as a viscous yellow oil.

ESI-MS m/z 458 (M+H)⁺.

¹H NMR (DMSO-d₆) 2 rotamers in a 1/1 ratio, each chemical shift is for both rotamers except when stated, δ (ppm): 8.52-8.48 (m, 1H), 7.99-7.97 (m, 1H), 7.83-7.76 (m, 2H), 7.57-7.28 (m, 10H), 5.13 (s, 2H, one rotamer), 4.86 (s, 2H, one rotamer), 4.83 (s, 2H, one rotamer), 4.77 (s, 2H, one rotamer), 4.58 (s, 2H, one rotamer), 4.56 (s, 2H, one rotamer), 4.46 (s, 2H, one rotamer), 4.07 (s, 2H, one rotamer).

### EXAMPLE II

### [{[5-(morpholin-4-ylmethyl)-2-phenyl-1,3-oxazol-4-yl]carbonyl}(pyridin-2-ylmethyl)amino]acetic Acid

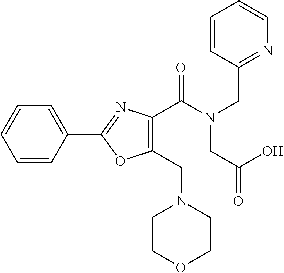

a)

To a mixture of ethyl 2-(benzoylamino)-4-chloro-3-oxobutanoate (415 mg, 1.46 mmol, prepared from ethyl 4-chloro-acetoacetate following the same procedure as in example I) in chloroform was added phosphorus oxychloride (120 μL, 0.240 mmol). The reaction mixture was stirred under argon and refluxed at 90° C. overnight. An aqueous solution of sodium hydrogen carbonate was added at 0° C. and after stirring for 0.5 h the reaction mixture was extracted with dichloromethane. The combined organic extracts were dried over sodium sulfate, filtered and evaporated. The crude product was purified by flash chromatography (silica gel, cyclohexane/ethyl acetate 95/5) to afford ethyl 5-(chloromethyl)-2-phenyl-1,3-oxazole-4-carboxylate (164 mg, 42%) as a beige solid.

ESI-MS m/z 266 and 268 (M+H)⁺.

¹H NMR (CDCl₃), δ (ppm): 8.18 (d, J=6.9 Hz, 2H), 7.55-7.53 (m, 3H), 5.07 (s, 2H), 4.52 (q, J=7.2 Hz, 2H), 1.50 (t; J=7.2 Hz, 3H).

b)

According to the experimental procedure used in example I, saponification of ethyl 5-(chloromethyl)-2-phenyl-1,3-oxazole-4-carboxylate (430 mg, 1.62 mmol) led to 5-(chloromethyl)-2-phenyl-1,3-oxazole-4-carboxylic acid (353.5 mg, 91%) as a white solid.

ESI-MS m/z 238 and 240 (M+H)⁺.

c)

To a mixture of 5-(chloromethyl)-2-phenyl-1,3-oxazole-4-carboxylic acid in dichloromethane cooled to 0° C. was added oxalyl chloride (120 μL, 0.24 mmol, 2M in dichloromethane) and dimethylformamide (1 drop). After stirring at room temperature for 2 h, methyl [(pyridine-2-ylmethyl)amino]acetate (32 mg, 0.176 mmol, prepared as described above) and N,N-diisopropylethylamine (84 μL, 0.480 mmol) were added. The reaction mixture was stirred at room temperature overnight. An aqueous solution of diluted hydrochloric acid (2 mL, 1N) was added and after stirring for 10 minutes the reaction mixture was extracted with dichloromethane. The combined organic extracts were dried over sodium sulfate, filtered and evaporated to afford methyl [{[5-(chloromethyl)-2-phenyl-1,3-oxazol-4-yl]carbonyl}(pyridin-2-ylmethyl)amino]acetate as an oil (62 mg, 97%). The crude product was used in the next reaction without purification.

ESI-MS m/z 400 and 402 (M+H)⁺.

d)

A mixture of methyl [{[5-(chloromethyl)-2-phenyl-1,3-oxazol-4-yl]carbonyl}(pyridin-2-ylmethyl)amino]acetate (60 mg, 0.15 mmol) and morpholine (44 μL, 0.5 mmol) in dichloromethane was stirred under argon at room temperature overnight. Water was added and the reaction mixture was extracted with dichloromethane. The combined organic extracts were dried over sodium sulfate, filtered and evaporated. The crude product, methyl [{[5-(morpholin-4-ylmethyl)-2-phenyl-1,3-oxazol-4-yl]carbonyl}(pyridin-2-ylmethyl)amino]acetate 50 mg, 75%) was engaged in the next reaction without purification.

ESI-MS m/z 451 (M+H)⁺.

e)

According to the experimental procedure used in example I, saponification of methyl [{[5-(morpholin-4-ylmethyl)-2-phenyl-1,3-oxazol-4-yl]carbonyl}(pyridin-2-ylmethyl)amino]acetate (50.4 mg, 0.12 mmol) led to [{[5-(morpholin-4-ylmethyl)-2-phenyl-1,3-oxazol-4-yl]carbonyl}(pyridin-2-ylmethyl)amino]acetic acid (17 mg, 35%) as a white solid.

ESI-MS m/z 437 (M+H)⁺.

¹H NMR (DMSO-d₆) 2 rotamers in a 3/2 ratio, each chemical shift is for both rotamers except when stated, δ (ppm): 8.52-8.47 (m, 1H), 7.97 (br s, 2H, major rotamer), 7.83-7.76 (m, 2H, minor rotamer, 1H), 7.55-7.52 (m, 3H), 7.40-7.34 (m, 1H), 7.30-7.25 (m, 1H), 5.07 (br s, 2H, minor rotamer), 4.75 (br s, 2H, major rotamer), 4.14 (br s, 2H, major rotamer), 3.86 (br s, 4H, minor rotamer), 3.55-3.20 (m, 2H), 2.69-2.65 (m, 2H).

### EXAMPLE III

### [{[2-(3-methoxyphenyl)-4-methyl-1,3-oxazol-5-yl]carbonyl}(pyridin-2-ylmethyl)amino]acetic Acid

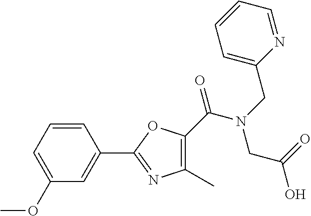

a)

A solution of ethyl 2-chloroacetoacetate (1.45 mL, 10 mmol) and 3-methoxybenzamide (1.55 g, 10 mmol) in anhydrous toluene (3 mL) was stirred at 120° C. for 2 hours, next at 140° C. for 2 hours and then at 120° C. overnight. An aqueous solution of ammonium chloride was added and the reaction mixture was extracted with ethyl acetate. The combined organic extracts were dried over sodium sulfate, filtered and evaporated. Purification by flash chromatography (silica gel, cyclohexane/ethyl acetate 95/5) to afforded ethyl 2-(3-methoxyphenyl)-4-methyl-1,3-oxazole-5-carboxylate (1.28 g, 48%) as a white solid.

ESI-MS m/z 262 (M+H)⁺.

b)

According to the experimental procedure used in example I, saponification of ethyl 2-(3-methoxyphenyl)-4-methyl-1,3-oxazole-5-carboxylate (1 g, 3.83 mmol) led to 2-(3-methoxyphenyl)-4-methyl-1,3-oxazole-5-carboxylic acid (845 mg, 94%) as a white solid. ESI-MS m/z 234 (M+H)⁺.

c)

According to the experimental procedure used in example I, the reaction between 2-(3-methoxyphenyl)-4-methyl-1,3-oxazole-5-carboxylic acid (130 mg, 0.56 mmol) and methyl [(pyridin-2-ylmethyl)amino]acetate (121 mg, 0.67 mmol, prepared as described previously) afforded methyl [{[2-(3-methoxyphenyl)-4-methyl-1,3-oxazol-5-yl]carbonyl}(pyridin-2-ylmethyl)amino]acetate (101 mg, 46%) as an oil.

ESI-MS m/z 396 (M+H)⁺.

d)

According to the experimental procedure used in example I, saponification of methyl [{[2-(3-methoxyphenyl)-4-methyl-1,3-oxazol-5-yl]carbonyl}(pyridin-2-ylmethyl)amino]acetate (59 mg, 0.15 mmol) led to [{[2-(3-methoxyphenyl)-4-methyl-1,3-oxazol-5-yl]carbonyl}(pyridin-2-ylmethyl)amino]acetic acid (48 mg, 84%) as a white solid.

ESI-MS m/z 382 (M+H)⁺.

¹H NMR (DMSO-d₆) 2 rotamers in a 2/1 ratio, each chemical shift is for both rotamers except when stated, δ (ppm): 8.74 (d, J=5.2 Hz, 1H, major rotamer), 8.71 (d, J=5.2 Hz, 1H, minor rotamer), 8.30-8.26 (m, 1H, major rotamer), 8.15-8.11 (m, 1H, minor rotamer), 7.82 (d, J=8 Hz, 1H, major rotamer), 7.76 (d, J=8 Hz, 1H, minor rotamer), 7.72 (t, J=6.4 Hz, 1H, major rotamer), 7.62-7.60 (m, 1H, minor rotamer, 1H, major rotamer), 7.51 (br s, 1H, major rotamer), 7.48 (t, J=7.8 Hz, 1H, major rotamer), 7.29 (t, J=7.8 Hz, 1H, minor rotamer), 7.17 (dd, J=8.4 Hz and J=2 Hz, 1H, major rotamer), 7.07 (dd, J=8.4 Hz and J=2 Hz, 1H, minor rotamer), 7.00 (br s, 1H, minor rotamer), 6.93 (d, J=7.6 Hz, 1H, minor rotamer), 5.14 (s, 1H, minor rotamer), 4.98 (s, 1H, major rotamer), 4.67 (s, 1H, major rotamer), 4.20 (s, 1H, minor rotamer), 3.85 (s, 3H, major rotamer), 3.72 (s, 3H, minor rotamer), 2.42 (s, 3H).

### EXAMPLE IV

### [({2-[3-(acetyloxy)phenyl]-4-methyl-1,3-oxazol-5-yl}carbonyl)(pyridin-2-ylmethyl)amino]acetic Acid

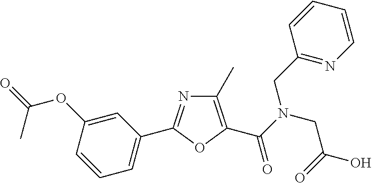

a)

Under argon at −78° C., to a solution of 2-(3-methoxyphenyl)-4-methyl-1,3-oxazole-5-carboxylic acid (100 mg, 0.42 mmol) in anhydrous dichloromethane (1.7 mL), was added boron tribromide (1M solution in dichloromethane, 1.3 mL, 1.3 mmol). The reaction mixture was stirred allowing the temperature to raise to −15° C. over a period of 2.5 h. An aqueous solution of potassium sodium tartrate was added and the temperature let to rise. The reaction mixture was acidified with aqueous hydrochloric acid, diluted with dichloromethane, and filtered. The white solid was collected and diluted with ethyl acetate and the organic solution was washed with aqueous hydrochloric acid. The combined organic extracts were dried over sodium sulfate, filtered and evaporated to afford 110 mg of a white solid.

Under argon, dichloromethane (4 mL) was added to the solid and the suspension was cooled to 0° C. Acetic anhydride (800 μL) and pyridine (1 mL) were successively added and the resulting mixture was kept stirring for 1.5 h, allowing the temperature to rise. An aqueous solution of sodium bicarbonate was added and the reaction mixture was extracted with dichloromethane. The combined organic extracts were dried over sodium sulfate, filtered and evaporated. The crude product was purified by preparative TLC (silica gel, dichloromethane/methanol 9/1) to afford 2-[3-(acetyloxy)phenyl]-4-methyl-1,3-oxazole-5-carboxylic acid (62.4 mg, 56%) as a beige solid.

b)

Under argon, a solution of 1-pyridin-2-ylmethanamine (625 μL, 6 mmol), benzyl chloroacetate (920 μL, 6 mmol) and triethylamine (916 μL, 6 mmol), in anhydrous N,N-dimethylformamide (12 mL) was stirred at 45° C. for 7 hours, then at room temperature for 2 days. An aqueous solution of sodium chloride was added and the reaction mixture was extracted with ethyl acetate. The combined organic extracts were dried over sodium sulfate, filtered and evaporated. The crude product was purified by flash chromatography (silica gel, dichloromethane/methanol 98/2) to afford benzyl [(pyridin-2-ylmethyl)amino]acetate (1.45 g, 74%) as a yellow oil.

¹H NMR (CDCl₃), δ (ppm): 8.55 (d, J=4.4 Hz, 1H), 7.64 (td, J=7.6 Hz and 1.6 Hz, 1H), 7.35-7.30 (m, 6H), 7.17-7.14 (m, 1H), 5.17 (s, 2H), 3.96 (s, 2H), 3.54 (s, 2H).

c)

According to the experimental procedure used in example I, the reaction between 2-[3-(acetyloxy)phenyl]-4-methyl-1,3-oxazole-5-carboxylic acid (62 mg, 0.24 mmol) and benzyl [(pyridin-2-ylmethyl)amino]acetate (67.4 mg, 0.26 mmol) afforded benzyl ({2-[3-(acetyloxy)phenyl]-4-methyl-1,3-oxazol-5-yl}carbonyl)(pyridin-2-ylmethyl)amino]acetate (56.6 mg, 47%).

To a solution of ({2-[3-(acetyloxy)phenyl]-4-methyl-1,3-oxazol-5-yl}carbonyl)(pyridin-2-ylmethyl)amino]acetate in degassed methanol (1 mL), was added palladium on activated charcoal (25 mg) and the reaction mixture was stirred at room temperature under hydrogen pressure (6 bar) for 2 days. The reaction mixture was then filtered through a pad of celite, rinsed with dichloromethane, and solvents were evaporated. Purification by preparative TLC (silica gel, dichloromethane/methanol/acetic acid 90/10/1) led to [({2-[3-(acetyloxy)phenyl]-4-methyl-1,3-oxazol-5-yl}carbonyl)(pyridin-2-ylmethyl)amino]acetic acid (10 mg, 21%) as an oil.

ESI-MS m/z 410 (M+H)⁺.

¹H NMR (CD₃OD) 2 rotamers in a 1/1 ratio, each chemical shift is for both rotamers except when stated, δ (ppm): 8.49-8.48 (m, 1H, one rotamer), 8.43-8.42 (m, 1H, one rotamer), 7.87-7.81 (m, 2H: 1H of both rotamers and 1H of one rotamer), 7.71-7.70 (m, 1H, one rotamer), 7.49-7.43 (m, 2H: 1H of both rotamers and 1H of one rotamer), 7.31-7.28 (m, 2H), 7.19 (d, J=7.6 Hz, 1H, one rotamer), 7.09 (dd, J=0.4 Hz and 6.8 Hz, 1H, one rotamer), 6.92 (br s, 1H, one rotamer), 4.96-4.94 (m, 2H, one rotamer), 4.83-4.81 (m, 2H, one rotamer), 4.47-4.45 (m, 2H, one rotamer), 4.23-4.21 (m, 2H, one rotamer), 2.37 (s, 3H), 2.22 (s, 3H).

In the following examples (example V and example VI), the carboxylic acids used in the amide bond formation reactions are prepared according to the experimental procedure used to prepare 2-(3-methoxyphenyl)-4-methyl-1,3-oxazole-5-carboxylic acid in example III.

### EXAMPLE V

### [{[2-(4-chlorophenyl)-4-methyl-1,3-oxazol-5-yl]carbonyl}(pyridin-2-ylmethyl)amino]acetic acid

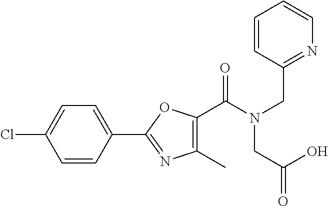

2-(4-chlorophenyl)-4-methyl-1,3-oxazole-5-carboxylic acid was prepared from 4-chlorobenzoic acid (1.59 g, 10 mmol) and ethyl 2-chloro-3-oxobutanoate (1.38 mL, 10 mmol) following the same experimental procedure as in example III.

a)

A mixture of 2-(4-chlorophenyl)-4-methyl-1,3-oxazole-5-carboxylic acid (50 mg, 0.21 mmol), N-(3-dimethylaminopropyl)-N′-ethylcarbodiimide hydrochloride (81 mg, 0.42 mmol), 4-dimethylaminopyridine (103 mg, 0.84 mmol) and methyl [(pyridin-2-ylmethyl)amino]acetate (46.0 mg, 0.25 mmol, prepared as described above) in dimethylformamide was stirred under argon at room temperature for 0.5 h and then at 50° C. overnight. An aqueous solution of ammonium chloride was added and the reaction mixture was extracted with ethyl acetate. The combined organic extracts were dried over sodium sulfate, filtered and evaporated. The crude product was purified by preparative TLC (silica gel, dichloromethane/methanol 95/5) to afford methyl [{[2-(4-chlorophenyl)-4-methyl-1,3-oxazol-5-yl]carbonyl}(pyridin-2-ylmethyl)amino]acetate (40 mg, 46%) as a solid.

b)

According to the experimental procedure used in example I, saponification of methyl [{[2-(4-chlorophenyl)-4-methyl-1,3-oxazol-5-yl]carbonyl}(pyridin-2-ylmethyl)amino]acetate (40 mg, 0.1 mmol) led to [{[2-(4-chlorophenyl)-4-methyl-1,3-oxazol-5-yl]carbonyl}(pyridin-2-ylmethyl)amino]acetic acid (13.5 mg, 35%) as a white solid.

¹H NMR (DMSO-d₆) 2 rotamers in a 3/2 ratio, each chemical shift is for both rotamers except when stated, δ (ppm): 8.27 (br s, 1H, major rotamer), (8.13 (br s, 1H, minor rotamer), 8.00 (d, J=8 Hz, 2H), 8.15-8.09 (m, 1H, minor rotamer), 7.81-7.87 (m, 1H, major rotamer), 7.64 (d, J=8 Hz, 2H), 7.50 (d, J=8 Hz, 1H), 7.38 (d, J=8 Hz, 1H), 5.13 (s, 2H, minor rotamer), 4.97 (s, 2H, major rotamer), 4.65 (s, 2H, major rotamer), 4.23 (s, 2H, minor rotamer), 2.41 (s, 3H).

### EXAMPLE VI

### [[(4-methyl-2-phenyl-1,3-oxazol-5-yl)carbonyl](pyridin-2-ylmethyl)amino]acetic Acid

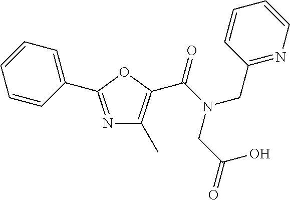

a)

A mixture of 4-methyl-2-phenyl-1,3-oxazole-5-carboxylic acid (203 mg, 1 mmol, prepared according to J. Chem. Soc. Perkin Trans. 1 1991, 2417), N-(3-dimethylaminopropyl)-N′-ethylcarbodiimide hydrochloride (383 mg, 2 mmol), 4-dimethylaminopyridine (367 mg, 3 mmol) and ethyl [(pyridin-2-ylmethyl)amino]acetate (207 mg, 1.07 mmol, prepared as in Heterocycles 1985, 349) in dichloromethane (10 mL) was stirred under argon at room temperature for 0.5 h and then at 50° C. overnight. An aqueous solution of ammonium chloride was added and the reaction mixture was extracted with dichloromethane. The combined organic extracts were dried over sodium sulfate, filtered and evaporated. The crude product was purified by flash chromatography (silica gel, dichloromethane/methanol 99/1 to 98/2) to afford ethyl [[(4-methyl-2-phenyl-1,3-oxazol-5-yl)carbonyl](pyridin-2-ylmethyl)amino]acetate (351 mg, 92%) as an oil.

ESI-MS m/z 380 (M+H)⁺.

b)

A mixture of ethyl [[(4-methyl-2-phenyl-1,3-oxazol-5-yl)carbonyl](pyridin-2-ylmethyl)amino]acetate (222 mg, 0.59 mmol) and lithium hydroxide (28 mg, 1.17 mmol) in tetrahydrofuran (4 mL) and water (4 mL) was stirred at room temperature overnight. The reaction mixture was then concentrated to give a white solid. To this solid, diluted aqueous hydrochloric acid and ethyl acetate were added and the suspension was stirred at room temperature overnight. The solid was then filtered and rinsed with water and ethyl acetate to give the title compound (190 mg, 92%) as a white solid.

ESI-MS m/z 352 (M+H)⁺.

¹H NMR (DMSO-d₆) 2 rotamers in a 1/1 ratio, each chemical shift is for both rotamers except when stated, δ (ppm): 8.64 (d, J=3.9 Hz, 1H, one rotamer), 8.59 (d, J=4.5 Hz, 1H, one rotamer), 8.05 (d, J=6.3 Hz, 2H, one rotamer), 7.91-7.89 (m, 2H, one rotamer), 7.61-7.40 (m, 6H), 5.05 (s, 2H, one rotamer), 4.84 (s, 2H, one rotamer), 4.60 (s, 2H, one rotamer), 4.25 (s, 2H, one rotamer), 2.45 (s, 3H, one rotamer), 2.43 (s, 3H, one rotamer).

In the following examples (example VII to example XXIII), the title compounds are prepared from carboxylic acids which are commercially available starting materials or readily prepared according to literature procedures, and from methyl [(pyridin-2-ylmethyl)amino]acetate prepared according to Bull. Chem. Soc. Jpn. 2002, 2423, following the representative procedures for the coupling of carboxylic acids with secondary amines and for saponification of esters as described in example I.

### EXAMPLE VII

### [(5-phenyl-2-furoyl)(pyridin-2-ylmethyl)amino]acetic Acid

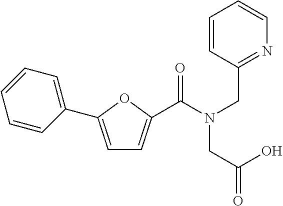

ESI-MS m/z 337 (M+H)⁺.

¹H NMR (DMSO-d₆) 2 rotamers in a 2/1 ratio, each chemical shift is for both rotamers except when stated, δ (ppm): 8.59 (br s, 1H, minor rotamer), 8.50 (br s, 1H, major rotamer), 7.82-7.69 (m, 2H), 7.46-7.26 (m, 6H), 7.13-7.04 (m, 2H), 5.01 (br s, 1H, minor rotamer), 4.74 (br s, 2H, major rotamer), 4.10 (br s, 2H, major rotamer), 3.97 (br s, 2H, minor rotamer).

### EXAMPLE VIII

### [[(1-methyl-3-phenyl-1H-pyrazol-5-yl)carbonyl](pyridin-2-ylmethyl)amino]acetic Acid

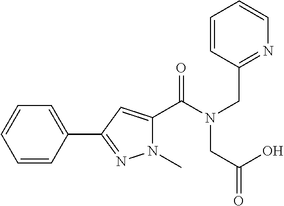

ESI-MS m/z 351 (M+H)⁺.

¹H NMR (DMSO-d₆) 2 rotamers in a 1/1 ratio, each chemical shift is for both rotamers except when stated, δ (ppm): 8.58 (d, 1H, J=3.6 Hz, one rotamer), 8.55 (d, 1H, J=4.4 Hz, one rotamer), 7.82-7.64 (m, 3H), 7.47-7.28 (m, 5H), 6.95 (br s, 1H, one rotamer), 6.82 (br s, 1H, one rotamer), 4.79 (br s, 2H), 4.24 (br s, 2H, one rotamer), 4.12 (br s, 2H, one rotamer), 3.88 (br s, 3H).

### EXAMPLE IX

### [[(4-methyl-2-phenyl-1,3-thiazol-5-yl)carbonyl](pyridin-2-ylmethyl)amino]acetic Acid

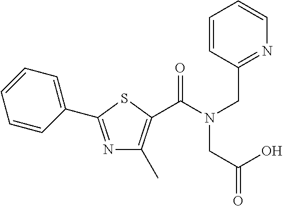

ESI-MS m/z 368 (M+H)⁺.

¹H NMR (DMSO-d₆) 2 rotamers in a 2/1 ratio, each chemical shift is for both rotamers except when stated, δ (ppm): 8.56-8.50 (m, 1H), 7.94-7.84 (m, 2H), 7.82-7.72 (m, 1H), 7.49 (br s, 3H), 7.38-7.34 (m, 1H, major rotamer), 7.32-7.22 (m, 1H, minor rotamer, 1H, both rotamers), 4.75 (br s, 2H, major rotamer), 4.70 (br s, 2H, minor rotamer), 3.92 (br s, 2H, minor rotamer), 3.68 (br s, 2H, major rotamer), 2.48 (s, 3H, minor rotamer), 2.42 (s, 3H, major rotamer).

### EXAMPLE X

### [(2-methyl-5-phenyl-3-furoyl)(pyridin-2-ylmethyl)amino]acetic Acid

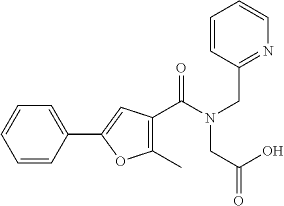

ESI-MS m/z 351 (M+H)⁺.

¹H NMR (DMSO-d₆) 2 rotamers in a 2/1 ratio, each chemical shift is for both rotamers except when stated, δ (ppm): 8.54 (br s, 1H, minor rotamer), 8.48 (br s, 1H, major rotamer), 7.80-7.73 (m, 1H), 7.64-7.55 (m, 2H), 7.42-7.24 (m, 5H), 6.97 (s, 1H, major rotamer), 6.84 (s, 1H, minor rotamer), 4.73 (br s, 2H), 3.89 (br s, 2H, minor rotamer), 3.79 (br s, 2H, major rotamer), 2.48 (br s, 3H).

### EXAMPLE XI

### [[(5-methyl-2-phenyl-2H-1,2,3-triazol-4-yl)carbonyl](pyridin-2-ylmethyl)amino]acetic Acid

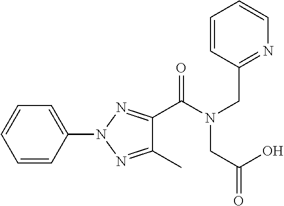

ESI-MS m/z 352 (M+H)⁺.

¹H NMR (DMSO-d₆) 2 rotamers in a 3/2 ratio, each chemical shift is for both rotamers except when stated, δ (ppm): 8.54-8.51 (m, 1H), 8.00 (d, 2H, J=7.6 Hz, major rotamer), 7.80 (t, 2H, J=7.6 Hz), 7.75 (d, 2H, J=7.6 Hz, minor rotamer), 7.58 (m, 4H), 7.32-7.28 (m, 1H), 5.03 (br s, 2H, minor rotamer), 4.82 (br s, 2H, major rotamer), 4.42 (br s, 2H, major rotamer), 4.16 (br s, 2H, minor rotamer), 2.46 (s, 3H, major rotamer), 2.43 (s, 3H, minor rotamer).

### EXAMPLE XII

### [[(5-methyl-2-phenyl-1,3-oxazol-4-yl)carbonyl](pyridin-2-ylmethyl)amino]acetic Acid

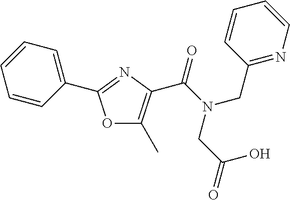

ESI-MS m/z 352 (M+H)⁺.

¹H NMR (DMSO-d₆) 2 rotamers in a 1/1 ratio, each chemical shift is for both rotamers except when stated, δ (ppm): 8.59-8.56 (m, 1H, one rotamer), 8.52-8.49 (m, 1H, one rotamer), 8.00-7.97 (m, 2H, one rotamer), 7.97-7.76 (m, 1H), 7.55-7.25 (m, 2H of one rotamer and 5H of both rotamers), 4.98 (s, 2H, one rotamer), 4.76 (s, 2H, one rotamer), 4.51 (s, 2H, one rotamer), 4.18 (s, 2H, one rotamer), 2.39 (s, 3H, one rotamer), 2.37 (s, 3H, one rotamer).

### EXAMPLE XIII

### [{[2-phenyl-5-(trifluoromethyl)-1,3-oxazol-4-yl]carbonyl}(pyridin-2-ylmethyl)amino]acetic Acid

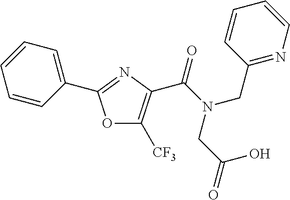

ESI-MS m/z 406 (M+H)⁺.

¹H NMR (CDCl₃) 2 rotamers in a 1/5 ratio, each chemical shift is for both rotamers except when stated, δ (ppm): 8.58-8.56 (m, 1H, minor rotamer), 8.52-8.50 (m, 1H, major rotamer), 8.13-8.08 (m, 2H, major rotamer), 7.92-7.85 (m, 1H of the major rotamer and 2H of the minor rotamer), 7.80-7.73 (m, 1H, minor rotamer), 7.53-7.47 (m, 4H), 7.41-7.36 (m, 1H), 5.09 (s, 2H, minor rotamer), 4.88 (s, 2H, major rotamer), 4.56 (s, 2H, major rotamer), 4.34 (s, 2H, minor rotamer).

### EXAMPLE XIV

### [({5-methyl-2-[3-(trifluoromethyl)phenyl]-1,3-oxazol-4-yl}carbonyl)(pyridin-2-ylmethyl)amino]acetic Acid

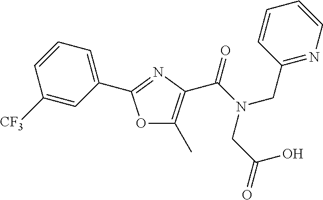

Purification by preparative HPLC after saponification afforded the trifluoroacetic salt of the title compound.

ESI-MS m/z 420 (M+H)⁺.

¹H NMR (CDCl₃) 2 rotamers in a roughly 1/3 ratio, each chemical shift is for both rotamers except when stated, δ (ppm): 9.45 (br s, 1H), 8.74 (br s, 1H), 8.30-8.25 (m, 1H), 8.20-8.15 (m, 2H, major rotamer), 8.02-7.95 (m, 1H), 7.85-7.52 (m, 2H, minor rotamer, 3H, both rotamers), 5.56 (s, 2H, minor rotamer), 5.10 (s, 2H, major rotamer), 4.90 (s, 2H, major rotamer), 4.34 (s, 2H, minor rotamer), 2.64 (s, 3H, minor rotamer), 2.61 (s, 3H, major rotamer).

### EXAMPLE XV

### [({5-methyl-2-[2-(trifluoromethyl)phenyl]-1,3-oxazol-4-yl}carbonyl)(pyridin-2-yl methyl)amino]acetic Acid

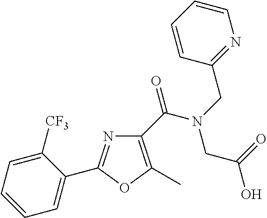

ESI-MS m/z 420 (M+H)⁺.

¹H NMR (CDCl₃) 2 rotamers in a roughly 2/3 ratio, each chemical shift is for both rotamers except when stated, δ (ppm): 8.56 (br s, 1H), 8.16 (d, J=8 Hz, 1H, major rotamer), 8.03-7.95 (m, 1H), 7.80-7.45 (m, 1H, minor rotamer, 6H, both rotamers), 5.64 (s, 2H, minor rotamer), 5.01 (s, 2H, major rotamer), 4.89 (s, 2H, major rotamer), 4.28 (s, 2H, minor rotamer), 2.66 (s, 3H, minor rotamer), 2.63 (s, 3H, major rotamer).

### EXAMPLE XVI

### [({5-methyl-2-[4-(trifluoromethyl)phenyl]-1,3-oxazol-4-yl}carbonyl)(pyridin-2-ylmethyl)amino]acetic Acid

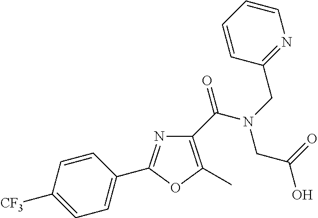

ESI-MS m/z 420 (M+H)⁺.

¹H NMR (CDCl₃) 2 rotamers in a roughly 1/3 ratio, each chemical shift is for both rotamers except when stated, δ (ppm): 8.77 (br s, 1H), 8.38-8.32 (m, 1H), 8.16-8.14 (m, 1H), 8.06 (d, J=8 Hz, 2H), 7.85-7.75 (m, 1H), 7.67 (d, J=8 Hz, 2H), 5.82 (s, 2H, minor rotamer), 5.35 (s, 2H, major rotamer), 5.10 (s, 2H, major rotamer), 4.42 (s, 2H, minor rotamer), 2.70 (s, 3H).

### EXAMPLE XVII

### [{[2-(4-bromophenyl)-5-methyl-1,3-oxazol-4-yl]carbonyl}(pyridin-2-ylmethyl)amino]acetic Acid

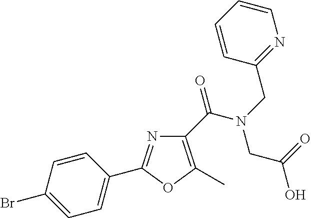

ESI-MS m/z 430 and 432 (M+H)⁺.

¹H NMR (CDCl₃) 2 rotamers in a roughly 1/3 ratio, each chemical shift is for both rotamers except when stated, δ (ppm): 8.51-8.50 (m, 1H), 7.93-7.89 (m, 3H), 7.60-7.53 (m, 3H), 7.42-7.37 (m, 1H), 5.50 (s, 2H, minor rotamer), 4.89 (s, 2H, major rotamer), 4.75 (s, 2H, major rotamer), 4.29 (s, 2H, minor rotamer), 2.61 (s, 3H).

### EXAMPLE XVIII

### [{[2-(3-bromophenyl)-5-methyl-1,3-oxazol-4-yl]carbonyl}(pyridin-2-ylmethyl)amino]acetic Acid

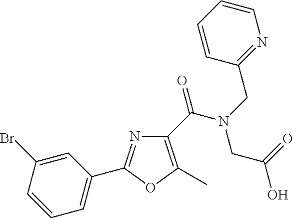

Purification by preparative HPLC after saponification afforded the trifluoroacetic salt of the title compound.

ESI-MS m/z 430 and 432 (M+H)⁺.

¹H NMR (CD₃OD) 2 rotamers in a roughly 1/2 ratio, each chemical shift is for both rotamers except when stated, δ (ppm): 8.73-8.68 (m, 1H), 8.38-8.32 (m, 1H), 8.17 (s, 1H, major rotamer), 8.01-7.95 (m, 2H, minor rotamer, 1H, both rotamers), 7.81-7.75 (m, 1H), 7.68-7.60 (m, 1H, major rotamer, 1H, both rotamers), 7.44 (t, J=8 Hz, 1H, major rotamer), 7.35 (t, J=8 Hz, 1H, minor rotamer), 5.39 (s, 2H, minor rotamer), 5.03 (s, 2H, major rotamer), 4.83 (s, 2H, major rotamer), 4.35 (s, 2H, minor rotamer), 2.66 (s, 3H, minor rotamer), 2.64 (s, 3H, major rotamer).

### EXAMPLE XIX

### [{[2-(2-bromophenyl)-5-methyl-1,3-oxazol-4-yl]carbonyl}(pyridin-2-ylmethyl)amino]acetic Acid

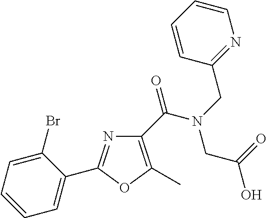

ESI-MS m/z 430 and 432 (M+H)⁺.

¹H NMR (CDCl₃) 2 rotamers in a roughly 1/1 ratio, each chemical shift is for both rotamers except when stated, δ (ppm): 8.48-8.43 (m, 1H), 8.02 (d, J=8 Hz, 1H, one rotamer), 7.82 (t, J=8 Hz, 1H, one rotamer), 7.70-7.59 (m, 2H), 7.51-7.38 (m, 1H, one rotamer, 1H, both rotamers), 7.35-7.16 (m, 1H, one rotamer, 2H, both rotamers), 5.39 (s, 2H, one rotamer), 4.85 (s, 2H, one rotamer), 4.78 (s, 2H, one rotamer), 4.08 (s, 2H, one rotamer), 2.61 (s, 3H, one rotamer), 2.56 (s, 3H, one rotamer).

### EXAMPLE XX

### [{[2-(3-methoxyphenyl)-5-methyl-1,3-oxazol-4-yl]carbonyl}(pyridin-2-ylmethyl)amino]acetic Acid

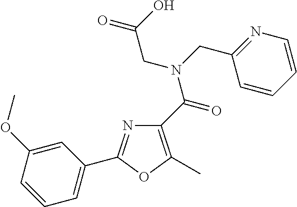

ESI-MS m/z 382 (M+H)⁺

¹H NMR (CDCl₃) 2 rotamers in a 1/3 ratio, each chemical shift is for both rotamers except when stated, δ (ppm): 8.52-8.48 (m, 1H), 7.88-7.83 (m, 1H), 7.64 (s, 1H), 7.59 (d, J=8 Hz, 1H), 7.51 (d, J=8 Hz, 1H), 7.37-7.30 (m, 2H), 7.01-6.98 (m, 1H), 5.47 (s, 2H, minor rotamer), 4.86 (s, 2H, major rotamer), 4.72 (s, 2H, major rotamer), 4.27 (s, 2H, minor rotamer), 3.94 (s, 3H, major rotamer), 3.83 (s, 3H, minor rotamer), 2.63 (s, 3H, minor rotamer), 2.61 (s, 3H, major rotamer).

### EXAMPLE XXI

### [{[2-(4-methoxyphenyl)-5-methyl-1,3-oxazol-4-yl]carbonyl}(pyridin-2-ylmethyl)amino]acetic Acid

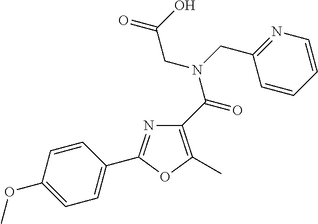

ESI-MS m/z 382 (M+H)⁺.

¹H NMR (CDCl₃) 2 rotamers in a roughly 1/3 ratio, each chemical shift is for both rotamers except when stated, δ (ppm): 8.47-8.46 (m, 1H), 7.94 (d, J=8 Hz, 2H, major rotamer), 7.82 (t, J=8 Hz, 1H, major rotamer), 7.74 (t, J=8 Hz, 1H, minor rotamer), 7.65 (d, J=8 Hz, 2H, minor rotamer), 7.49-7.46 (m, 1H), 7.32-7.23 (m, 1H), 6.94 (d, J=8 Hz, 2H, major rotamer), 6.86 (d, J=8 Hz, 2H, minor rotamer), 5.38 (s, 2H, minor rotamer), 4.84 (s, 2H, major rotamer), 4.70 (s, 2H, major rotamer), 4.17 (s, 2H, minor rotamer), 3.83 (s, 3H, major rotamer), 3.81 (s, 3H, minor rotamer), 2.57 (s, 3H, major rotamer), 2.54 (s, 3H, minor rotamer).

### EXAMPLE XXII

### [{[5-methyl-2-(2-nitrophenyl)-1,3-oxazol-4-yl]carbonyl}(pyridin-2-ylmethyl)amino]acetic Acid

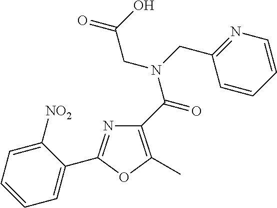

ESI-MS m/z 397 (M+H)⁺.

¹H NMR (CDCl₃) 2 rotamers in a roughly 2/3 ratio, each chemical shift is for both rotamers except when stated, δ (ppm): 8.54-8.53 (m, 1H), 8.13 (d, J=8 Hz, 1H, major rotamer), 7.95 (t, J=8 Hz, 1H), 7.83-7.79 (m, 1H, minor rotamer, 3H, both rotamers), 7.45-7.42 (m, 1H), 5.46 (s, 2H, minor rotamer), 4.93 (s, 2H, major rotamer), 4.79 (s, 2H, major rotamer), 4.24 (s, 2H, minor rotamer), 2.64 (s, 3H, minor rotamer), 2.57 (s, 3H, major rotamer).

### EXAMPLE XXIII

### [{[5-methyl-2-(3-nitrophenyl)-1,3-oxazol-4-yl]carbonyl}(pyridin-2-ylmethyl)amino]acetic Acid

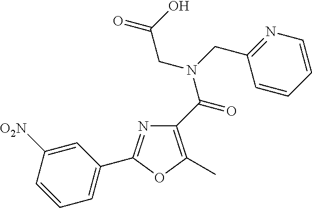

ESI-MS m/z 397 (M+H)⁺.

¹H NMR (CD₃OD) 2 rotamers in a roughly 1/1 ratio, each chemical shift is for both rotamers except when stated, δ (ppm): 8.76 (br s, 1H, one rotamer), 8.56-8.52 (m, 1H), 8.42 (br s, 1H, one rotamer), 8.39 (d, J=8 Hz, 1H, one rotamer), 8.34-8.27 (m, 1H), 8.11 (d, J=7.2 Hz, 1H, one rotamer), 7.95-7.87 (m, 1H), 7.75 (t, J=8 Hz, 1H, one rotamer), 7.69 (t, J=8 Hz, 1H, one rotamer), 7.65 (d, J=8 Hz, 1H, one rotamer), 7.55 (d, J=8 Hz, 1H, one rotamer), 7.41-7.35 (m, 1H), 5.26 (s, 2H, one rotamer), 4.93 (s, 2H, one rotamer), 4.61 (s, 2H, one rotamer), 4.23 (s, 2H, one rotamer), 2.67 (s, 3H).

### EXAMPLE XXIV

### [{[2-(4-hydroxyphenyl)-5-methyl-1,3-oxazol-4-yl]carbonyl}(pyridin-2-ylmethyl)amino]acetic Acid

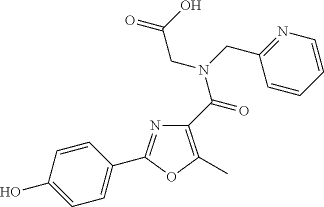

A solution of methyl [{[2-(4-methoxyphenyl)-5-methyl-1,3-oxazol-4-yl]carbonyl}(pyridin-2-ylmethyl)amino]acetate (60 mg, 0.15 mmol, prepared as in example XXI) in anhydrous dichloromethane (2 mL) was cooled in an ice bath, then boron tribromide (56 mg, 0.2 mmol) was added dropwise. The reaction mixture was then allowed to warm to room temperature and stirred overnight. The reaction mixture was quenched by addition of 5 mL of water and the layers were separated. The aqueous phase was freeze dried and the obtained residue was purified by preparative HPLC to get 12 mg (21%) of [{[2-(4-hydroxyphenyl)-5-methyl-1,3-oxazol-4-yl]carbonyl}(pyridin-2-ylmethyl)amino]acetic acid.

ESI-MS m/z 368 (M+H)⁺.

¹H NMR (CD₃OD), 2 rotamers in a 1/2 ratio, each chemical shift is for both rotamers except when stated, δ (ppm): 8.52 (br s, 1H), 7.94-7.91 (m, 1H), 7.87-7.85 (m, 1H), 7.66-7.58 (m, 2H), 7.43-7.40 (m, 1H), 6.87 (d, J=8 Hz, 2H, major rotamer), 6.80 (d, J=8 Hz, 2H, minor rotamer), 5.32 (s, 2H, minor rotamer), 4.69 (s, 2H, major rotamer), 4.24 (s, 2H, minor rotamer), 2.60 (s, 3H).

The following compound was prepared on a similar way:

### EXAMPLE XXV

### [{[2-(3-hydroxyphenyl)-5-methyl-1,3-oxazol-4-yl]carbonyl}(pyridin-2-ylmethyl)amino]acetic Acid

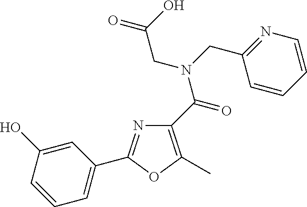

ESI-MS m/z 368 (M+H)⁺.

¹H NMR (CD₃OD), 2 rotamers in a 1/3 ratio, each chemical shift is for both rotamers except when stated, δ (ppm): 8.51 (br s, 1H), 7.88-7.86 (m, 1H), 7.62-7.44 (m, 3H), 7.37-7.20 (m, 2H), 6.90-6.89 (m, 1H), 5.28 (s, 1H, minor rotamer), 4.92 (s, 2H, major rotamer), 4.54 (s, 2H, major rotamer), 4.12 (s, minor rotamer), 2.60 (s, 3H).

### EXAMPLE XXVI

### [{[2-(2-aminophenyl)-5-methyl-1,3-oxazol-4-yl]carbonyl}(pyridin-2-ylmethyl)amino]acetic Acid

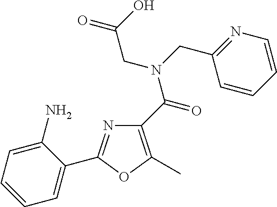

To a solution of [{[5-methyl-2-(2-nitrophenyl)-1,3-oxazol-4-yl]carbonyl}(pyridin-2-ylmethyl)amino]acetic acid (40 mg, 0.1 mmol) in dry methanol (4 mL), was added ferric chloride (2 mg, 5% by weight) and activated charcoal (2 mg, 5% by weight). The reaction mixture was heated to 65° C. Hydrazine hydrate (40 mg, 0.8 mmol) was added dropwise. The reaction mixture was refluxed overnight and then cooled to room temperature. Then the reaction mixture was filtered through a pad of celite and the filtrate was concentrated. Purification of the crude product by preparative HPLC afforded 15 mg (40%) of [{[2-(2-aminophenyl)-5-methyl-1,3-oxazol-4-yl]carbonyl}(pyridin-2-ylmethyl)amino]acetic acid as its trifluoroacetic acid salt.

ESI-MS m/z 367 (M+H)⁺.

¹H NMR (CD₃OD), 2 rotamers in a 1/2 ratio, each chemical shift is for both rotamers except when stated, δ (ppm): 8.83-8.78 (m, 1H), 8.56-8.52 (m, 1H), 8.19 (d, J=8 Hz, 1H, major rotamer), 8.13 (d, J=8 Hz, 1H, minor rotamer), 7.97-7.92 (m, 1H), 7.79 (d, J=8 Hz, 1H, major rotamer), 7.68 (d, J=8 Hz, 1H, minor rotamer), 7.24 (t, J=8 Hz, 1H, major rotamer), 7.15 (t, J=8 Hz, minor rotamer), 6.91 (d, J=8 Hz, 1H, major rotamer), 6.78 (t, J=8 Hz, 1H, major rotamer), 6.72-6.68 (m, 2H, minor rotamer), 5.42 (s, minor rotamer), 5.08 (s, 2H, major rotamer), 4.87 (s, 2H, major rotamer), 4.31 (s, minor rotamer), 2.63 (s, minor rotamer), 2.59 (s, 3H, major rotamer).

The following compound was prepared on a similar way:

### EXAMPLE XXVII

### [{[2-(4-aminophenyl)-5-methyl-1,3-oxazol-4-yl]carbonyl}(pyridin-2-ylmethyl)amino]acetic Acid

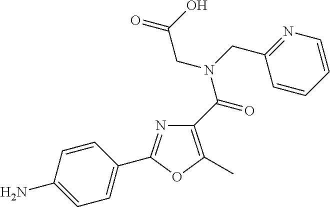

ESI-MS m/z 367 (M+H)⁺.

¹H NMR (CD₃OD), 2 rotamers in a roughly 3/2 ratio, each chemical shift is for both rotamers except when stated, δ (ppm): 8.53 (br s, 1H), 7.95-7.91 (m, 1H), 7.73 (d, J=8 Hz, 2H, major rotamer), 7.65 (d, J=8 Hz, 1H, minor rotamer), 7.59 (d, J=8 Hz, 1H, major rotamer), 7.49 (d, J=8 Hz, 2H, minor rotamer), 7.43-7.39 (m, 1H), 6.72 (d, J=8 Hz, 2H, major rotamer), 6.65 (d, J=8 Hz, 2H, minor rotamer), 5.32 (s, 2H, minor rotamer), 4.87 (s, 2H, major rotamer), 4.70 (s, 2H, major rotamer), 4.24 (s, 2H, minor rotamer), 2.58 (s, 3H, major rotamer), 2.56 (s, 3H, minor rotamer).

### EXAMPLE XXVIII

### {[(2-{3-[(cyclopropylcarbonyl)amino]phenyl}-5-methyl-1,3-oxazol-4-yl)carbonyl](pyridin-2-ylmethyl)amino}acetic Acid

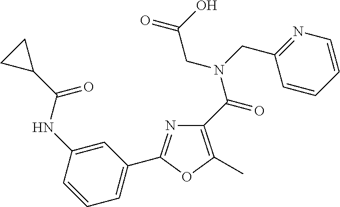

a)

Under argon, to a solution of methyl [{[2-(3-aminophenyl)-5-methyl-1,3-oxazol-4-yl]carbonyl}(pyridin-2-ylmethyl)amino]acetate (50 mg, 0.13 mmol, prepared as in example XXVII by reduction of the nitro compound synthesized as in example XXIII), and triethylamine (40 mg, 0.4 mmol) in dry dichloromethane (2 mL) was cooled to 0° C., cyclopropanecarbonyl chloride (20 mg, 0.2 mmol) was added. The reaction mixture was allowed to warm to room temperature and stirred at room temperature for 30 min. The reaction was quenched with water. The layers were separated and the aqueous layer was extracted with dichloromethane. The combined organic layers were washed with brine, dried over anhydrous sodium sulfate, filtered and concentrated. The crude product purified by chromatography (silica gel, dichloromethane/methanol 9/1) to obtain methyl {[(2-{3-[(cyclopropylcarbonyl)amino]phenyl}-5-methyl-1,3-oxazol-4-yl)carbonyl](pyridin-2-ylmethyl)amino}acetate (30 mg, 51%) as a colourless oil.

ESI-MS m/z 449 (M+H)⁺.

b)

According to the experimental procedure used in example I, saponification of methyl {[(2-{3-[(cyclopropylcarbonyl)amino]phenyl}-5-methyl-1,3-oxazol-4-yl)carbonyl](pyridin-2-ylmethyl)amino}acetate followed by purification by preparative HPLC led to {[(2-{3-[(cyclopropylcarbonyl)amino]phenyl}-5-methyl-1,3-oxazol-4-yl)carbonyl](pyridin-2-ylmethyl)amino}acetic acid as the TFA salt.

ESI-MS m/z 435 (M+H)⁺.

¹H NMR (CD₃OD), 2 rotamers in a roughly 1/1 ratio, each chemical shift is for both rotamers except when stated, δ (ppm): 8.83-8.79 (m, 1H, one rotamer), 8.78-8.72 (m, 1H, one rotamer), 8.49-8.41 (m, 1H), 8.31 (s, 1H, one rotamer), 8.23 (s, 1H, one rotamer), 8.11-8.07 (m, 1H), 7.89-7.84 (m, 1H), 7.77-7.75 (m, 1H, one rotamer), 7.65-7.63 (m, 1H, one rotamer), 7.46-7.35 (m, 2H), 5.47 (s, 2H, one rotamer), 5.05 (s, 2H, one rotamer), 4.34 (s, 2H, one rotamer), 2.66 (s, 3H, one rotamer), 2.62 (s, 3H, one rotamer), 1.81-1.78 (m, 1H), 1.00 (br s, 4H, one rotamer), 0.91 (br s, 4H, one rotamer).

### EXAMPLE XXIX

### ((1,3-benzothiazol-2-ylmethyl){[2-(3-methoxyphenyl)-4-methyl-1,3-oxazol-5-yl]carbonyl}amino)acetic Acid

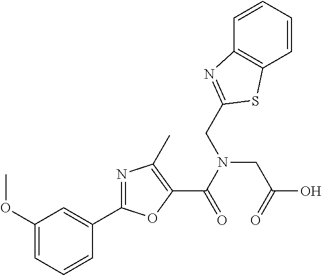

a)

Under argon, a solution of 1,3-benzothiazol-2-ylmethylamine hydrochloride (100 mg, 0.5 mmol), ethyl chloroacetate (54 μL, 0.5 mmol) and triethylamine (152 μL, 1.1 mmol) in anhydrous N,N-dimethylformamide (1 mL) was stirred at room temperature for 0.5 h, then at 50° C. overnight. Cold water was added and the reaction mixture was extracted with ethyl acetate. The combined organic extracts were dried over sodium sulfate, filtered and evaporated. The crude product was purified by preparative TLC (silica gel, dichloromethane/methanol 95/5) to afford ethyl [(1,3-benzothiazol-2-ylmethyl)amino]acetate (53.9 mg, 43%) as a yellow oil.

ESI-MS m/z 251 (M+H)⁺.

b)

According to the representative experimental procedures used in example I for the coupling of carboxylic acids with amines and for the saponification of esters, the reaction of 2-(3-methoxyphenyl)-4-methyl-1,3-oxazole-5-carboxylic acid with ethyl [(1,3-benzothiazol-2-ylmethyl)amino]acetate led to ((1,3-benzothiazol-2-ylmethyl){[2-(3-methoxyphenyl)-4-methyl-1,3-oxazol-5-yl]carbonyl}amino)acetic acid.

ESI-MS m/z 438 (M+H)⁺.

¹H NMR (CDCl₃) 2 rotamers in a roughly 1/3 ratio, each chemical shift is for both rotamers except when stated, δ (ppm): 8.04 (d, J=8.4 Hz, 1H, minor rotamer), 7.99 (d, J=8.1 Hz, 1H, major rotamer), 7.89 (d, J=7.5 Hz, 1H, major rotamer), 7.70 (d, J=7.8 Hz, 1H, major rotamer), 7.62 (s, 1H, major rotamer), 7.55-7.41 (m, 2H), 7.37-7.26 (m, 2H, minor rotamer, 1H, both rotamers), 7.18-7.12 (m, 1H, minor rotamer), 7.02 (dd, J=8.1 Hz and 1.8 Hz, 1H, major rotamer), 6.95-6.92 (m, 1H, minor rotamer), 5.40 (s, 2H, minor rotamer), 5.24 (s, 2H, major rotamer), 4.61 (s, 2H, major rotamer), 4.36 (s, 2H, minor rotamer), 3.87 (s, 3H, major rotamer), 3.60 (s, 3H, minor rotamer), 2.57 (3H).

### EXAMPLE XXX

### ((5-methoxy-1,3-benzothiazol-2-ylmethyl){[2-(3-methoxyphenyl)-4-methyl-1,3-oxazol-5-yl]carbonyl}amino)acetic Acid

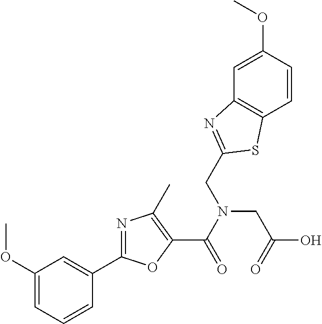

According to the representative experimental procedures used in example I for the coupling of carboxylic acids with amines and for the saponification of esters, the reaction of 2-(3-methoxyphenyl)-4-methyl-1,3-oxazole-5-carboxylic acid with ethyl [(5-methoxy-1,3-benzothiazol-2-ylmethyl)amino]acetate (prepared from (5-methoxy-1,3-benzothiazol-2-yl)methylamine as in example XXIX) led to ((5-methoxy-1,3-benzothiazol-2-ylmethyl){[2-(3-methoxyphenyl)-4-methyl-1,3-oxazol-5-yl]carbonyl}amino)acetic acid.

ESI-MS m/z 468 (M+H)⁺.

¹H NMR (CD₃OD) 2 rotamers in a roughly 3/4 ratio, each chemical shift is for both rotamers except when stated, δ (ppm): 7.89 (d, J=8.8 Hz, 1H, minor rotamer), 7.85 (d, J=8.8 Hz, 1H, major rotamer), 7.67 (d, J=7.6 Hz, 1H, major rotamer), 7.61 (s, 1H, major rotamer), 7.56-7.52 (m, 1H), 7.46 (t, J=8 Hz, 1H, major rotamer), 7.24-7.10 (m, 2H, minor rotamer, 2H, both rotamers), 7.01-6.99 (m, 1H, minor rotamer), 5.38 (s, 2H, minor rotamer), 5.18 (s, 2H, major rotamer), 4.67 (s, 2H, major rotamer), 4.43 (s, 2H, minor rotamer), 3.94 (s, 3H, minor rotamer), 3.92 (s, 6H, major rotamer), 3.59 (s, 3H, minor rotamer), 2.57 (s, 3H, major rotamer), 2.54 (s, 3H, minor rotamer).

### EXAMPLE XXXI

### {1-[(5-methyl-2-phenyl-1,3-oxazol-4-yl)carbonyl]-2-pyridin-2-ylhydrazino}acetic Acid

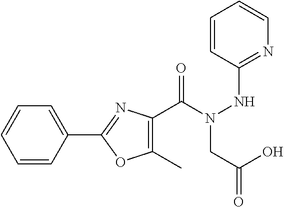

a)

Under argon, to a solution of 2-hydrazinopyridine (109 mg, 1 mmol) in dimethylformamide was added benzyl chloroacetate (152 μL, 1 mmol) and triethylamine (139 μL, 1 mmol). The reaction mixture was stirred overnight at 40° C. After cooling at room temperature, water was added and the reaction mixture was extracted with ethyl acetate. The combined organic extracts were dried over sodium sulfate, filtered and evaporated. The crude product was purified by flash chromatography (silica gel, dichloromethane/methanol 9/1) to afford 230 mg of benzyl (2-pyridin-2-ylhydrazino)acetate as a solid which was engaged in the next reaction. According to the experimental procedure used in example I for the coupling of carboxylic acids with amines, the reaction between benzyl (2-pyridin-2-ylhydrazino)acetate (50 mg, 0.19 mmol) and 5-methyl-2-phenyl-1,3-oxazole-4-carboxylic acid (47.4 mg, 0.23 mmol) gave after purification by preparative TLC (silica gel, cyclohexane/ethyl acetate 6/4) benzyl {1-[(5-methyl-2-phenyl-1,3-oxazol-4-yl)carbonyl]-2-pyridin-2-ylhydrazino}acetate (20.2 mg, 24%). ESI-MS m/z 443 (M+H)⁺.

b)

According to the experimental procedure used in example I, saponification of benzyl {1-[(5-methyl-2-phenyl-1,3-oxazol-4-yl)carbonyl]-2-pyridin-2-ylhydrazino}acetate led to {1-[(5-methyl-2-phenyl-1,3-oxazol-4-yl)carbonyl]-2-pyridin-2-ylhydrazino}acetic acid.

ESI-MS m/z 293 (M+H)⁺.

¹H NMR (CD₃OD) 2 rotamers in a 5/1 ratio, each chemical shift is for both rotamers except when stated, δ (ppm): 8.07 (br s, 2H, major rotamer), 7.80 (d, J=6 Hz, 2H, minor rotamer, 1H, both rotamers), 7.63 (t, J=7.6 Hz, 1H), 7.51 (br s, 1H), 7.43-7.41 (m, 2H), 6.90-6.80 (m, 2H), 2.66 (s, 3H, minor rotamer), 2.56 (s, 3H, major rotamer).

### EXAMPLE XXXII

### ([(4-methyl-2-phenyl-1,3-oxazol-5-yl)carbonyl]{[5-(2-fluorophenyl)-2-furyl]methyl}amino)acetic Acid

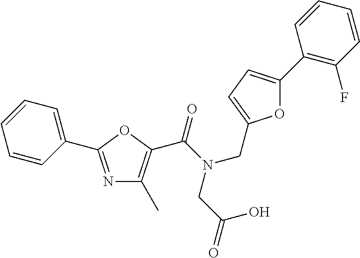

### a) Representative Procedure for Reductive Amination of Aldehydes:

Under argon, triethylamine (166 μL, 1.2 mmol) was added to a solution of 5-bromo-2-furaldehyde (180 mg, 1 mmol) and glycine methyl ester hydrochloride (152 mg, 1.2 mmol) in anhydrous dichloromethane (3 mL). The reaction mixture was stirred for 3 hours at room temperature, then sodium cyanoborohydride (1M in tetrahydrofuran, 1.5 mL, 1.5 mmol) was added and the reaction was kept stirring overnight. An aqueous solution of sodium bicarbonate was added and the reaction mixture was extracted with dichloromethane. The combined organic extracts were dried over sodium sulfate, filtered and evaporated. The crude product was purified by flash chromatography (silica gel, cyclohexane/ethyl acetate 50/50) to afford methyl {[(5-bromo-2-furyl)methyl]amino}acetate (187 mg, 76%) as an oil.

¹H NMR (CDCl₃), δ (ppm): 6.19 (d, J=3.0 Hz, 1H), 6.15 (d, J=3.0 Hz, 1H), 3.76 (s, 2H), 3.70 (s, 2H), 3.40 (s, 3H).

b)

According to the representative procedure used in example I for the coupling of carboxylic acids with amines, the reaction between 5-[(benzyloxy)methyl]-2-phenyl-1,3-oxazole-4-carboxylic acid (50 mg, 0.25 mmol) and methyl {[(5-bromo-2-furyl)methyl]amino}acetate (73 mg, 0.29 mmol) afforded methyl {[(5-bromo-2-furyl)methyl][(4-methyl-2-phenyl-1,3-oxazol-5-yl)carbonyl]amino}acetate (99 mg, 93%).

¹H NMR (CDCl₃+CD₃OD) 2 rotamers in a 1/1 ratio, each chemical shift is for both rotamers except when stated, δ (ppm): 7.96-7.92 (m, 2H), 7.47-7.46 (m, 3H), 6.34-6.27 (m, 2H), 4.82 (s, 2H, one rotamer), 4.72 (s, 2H, one rotamer), 4.45 (s, 2H, one rotamer), 4.17 (s, 2H, one rotamer), 3.76 (s, 3H), 2.51 (s, 3H).

c)

According to the experimental procedure used in example I, saponification of methyl {[(5-bromo-2-furyl)methyl][(4-methyl-2-phenyl-1,3-oxazol-5-yl)carbonyl]amino}acetate (93.4 mg, 0.2 mmol) led to {[(5-bromo-2-furyl)methyl][(4-methyl-2-phenyl-1,3-oxazol-5-yl)carbonyl]amino}acetic acid (56.1 mg, 62%) as a beige solid.

ESI-MS m/z 417 and 419 (M−H)⁻.

¹H NMR (DMSO-d₆) 2 rotamers in a 2/1 ratio, each chemical shift is for both rotamers except when stated, δ (ppm): 8.05-7.95 (m, 2H), 7.53 (Br s, 3H), 6.56-6.51 (m, 2H), 4.81 (s, 2H, minor rotamer), 4.66 (s, 2H, major rotamer), 4.20 (s, 2H, major rotamer), 3.95 (s, 2H, minor rotamer), 2.41 (s, 3H).

d)

Under argon, a solution of {[(5-bromo-2-furyl)methyl][(4-methyl-2-phenyl-1,3-oxazol-5-yl)carbonyl]amino}acetic acid (42 mg, 0.1 mmol), 2-fluorophenylboronic acid (28 mg, 0.2 mmol), cesium fluoride (62 mg, 0.4 mmol), and tetrakis(triphenylphosphine)palladium (8.8 mg, 0.008 mmol) in degassed methanol (0.5 mL) and toluene (0.5 mL) was stirred at 60° C. for 22 h. The reaction mixture was filtered through a bed of celite and rinsed with dichloromethane, methanol and ethyl acetate. The solvents were evaporated and the crude product was purified by preparative TLC (silica gel, dichloromethane/methanol 9/1) to give ([(4-methyl-2-phenyl-1,3-oxazol-5-yl)carbonyl]{[5-(2-fluorophenyl)-2-furyl]methyl}amino)acetic acid (8.6 mg, 20%) as a beige solid.

ESI-MS m/z 435 (M+H)⁺.

¹H NMR (DMSO-d₆) 2 rotamers in a 2/1 ratio, each chemical shift is for both rotamers except when stated, δ (ppm): 8.04-8.03 (m, 2H, major rotamer), 7.87-7.85 (m, 2H, minor rotamer), 7.78-7.75 (m, 1H, major rotamer), 7.66-7.64 (m, 1H, minor rotamer), 7.51 (Br s, 3H), 7.32-7.28 (m, 3H), 6.79 (br s, 1H), 6.55 (br s, 1H), 4.94-4.93 (m, 2H, minor rotamer), 4.76 (br s, 2H, major rotamer), 4.10 (br s, 2H, major rotamer), 3.94-3.92 (m, 2H, minor rotamer), 2.40 (s, 3H).

### EXAMPLE XXXIII

### [[(5-methyl-2-phenyl-1,3-oxazol-4-yl)carbonyl](1,3-thiazol-2-ylmethyl)amino]acetic Acid

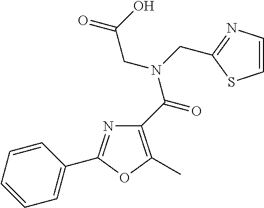

According to the representative experimental procedures used in example I for the coupling of carboxylic acids with amines and for the saponification of esters, the reaction of 5-methyl-2-phenyl-1,3-oxazole-4-carboxylic acid with ethyl [(1,3-thiazol-2-ylmethyl)amino]acetate (prepared from thiazole-2-carbaldehyde following the same representative procedure for reductive amination as in example XXXI) led to [[(5-methyl-2-phenyl-1,3-oxazol-4-yl)carbonyl](1,3-thiazol-2-ylmethyl)amino] acetic acid.

ESI-MS m/z 358 (M+H)⁺.

¹H NMR (CDCl₃) 2 rotamers in roughly 2/3 ratio, each chemical shift is for both rotamers except when stated, δ (ppm): 8.00-7.97 (m, 2H), 7.81-7.76 (m, 1H), 7.48-7.41 (br s, 4H), 5.66 (s, 2H, minor rotamer), 5.12 (s, 2H, major rotamer), 4.73 (s, 2H, major rotamer), 4.28 (s, 2H, minor rotamer), 2.72 (s, 3H, minor rotamer), 2.69 (s, 3H, major rotamer).

The following compounds were prepared on a similar way:

### EXAMPLE XXXIV

### [{[2-(3-methoxyphenyl)-5-methyl-1,3-oxazol-4-yl]carbonyl}(quinolin-2-ylmethyl)amino]acetic Acid

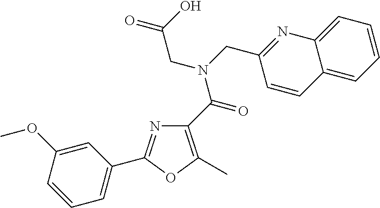

ESI-MS m/z 432 (M+H)⁺.

¹H NMR (CD₃OD), 2 rotamers in a roughly 3/2 ratio, each chemical shift is for both rotamers except when stated, δ (ppm): 9.01-8.97 (m, 1H), 8.30-8.21 (m, 2H), 8.15-8.07 (m, 2H), 7.92-7.88 (m, 1H), 7.62-7.60 (m, 2H, major rotamer), 7.42 (t, J=8 Hz, 1H, major rotamer), 7.19 (t, J=8 Hz, minor rotamer), 7.10-7.06 (m, 1H), 6.95-6.92 (m, 1H, minor rotamer), 6.85 (br s, 1H minor rotamer), 5.64 (s, 2H, minor rotamer), 5.25 (s, 2H, major rotamer), 4.97 (s, 2H, major rotamer), 4.48 (s, 2H, minor rotamer), 3.89 (s, 3H, major rotamer), 3.58 (s, 3H, minor rotamer), 2.66 (s, 3H, minor rotamer), 2.61 (s, 3H, major rotamer).

### EXAMPLE XXXV

### [{[2-(3-methoxyphenyl)-4-methyl-1,3-oxazol-5-yl]carbonyl}(1-pyridin-2-ylethyl)amino]acetic Acid

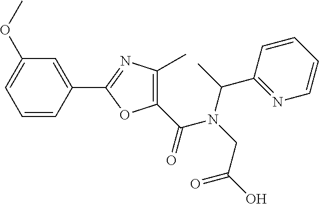

ESI-MS m/z 396 (M+H)⁺.

¹H NMR (DMSO-d₆) 2 rotamers in a 3/1 ratio, each chemical shift is for both rotamers except when stated, δ (ppm):

8.59-8.48 (m, 1H), 7.81-7.72 (m, 1H), 7.58 (d, J=8 Hz, 1H, major rotamer), 7.52-7.45 (m, 1H), 7.45-7.39 (m, 1H, major rotamer, 1H, both rotamers), 7.31-7.27 (m, 2H, minor rotamer, 1H, both rotamers), 7.10 (d, J=8 Hz, 1H), 5.83-5.75 (m, 1H, major rotamer), 5.61-5.58 (m, 1H, minor rotamer), 4.43-4.38 (m, 1H, minor rotamer), 4.14-4.09 (m, 1H, major rotamer), 3.83 (s, 3H, major rotamer), 3.78 (s, 3H, minor rotamer), 2.44 (s, 3H, major rotamer), 2.36 ‘(s, 3H, minor rotamer), 1.69 (d, J=7 Hz, 3H, minor rotamer), 1.55 (d, J=6.9 Hz, 3H, major rotamer).

### EXAMPLE XXXVI

### [{[2-(3-acetylphenyl)-4-methyl-1,3-thiazol-5-yl]carbonyl}(pyridin-2-ylmethyl)amino]acetic Acid

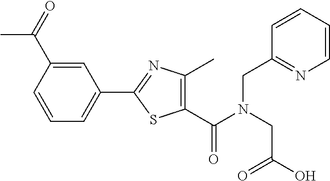

a)

Under argon, a solution of ethyl 2-bromo-4-methyl-1,3-thiazole-5-carboxylate (129 mg, 0.5 mmol, commercially available), 3-acetylphenylboronic acid (164 mg, 1 mmol), cesium carbonate (326 mg, 1 mmol), and tetrakis(triphenylphosphine)palladium (20.2 mg, 0.017 mmol) in degassed 1,4-dioxane (5 mL) was stirred at 85° C. for 24 h, then at 110° C. for 24 h. The reaction mixture was filtered through a bed of celite and rinsed with dichloromethane, methanol and ethyl acetate. The solvents were evaporated and the crude product was purified by preparative TLC (silica gel, cyclohexane/ethyl acetate 7/3) to give ethyl 2-(3-acetylphenyl)-4-methyl-1,3-thiazole-5-carboxylate (52.6 mg, 35%).

ESI-MS m/z 290 (M+H)⁺.

According to the experimental procedure used in example I, saponification of ethyl 2-(3-acetylphenyl)-4-methyl-1,3-thiazole-5-carboxylate (84.2 mg, 0.29 mmol) led to 2-(3-acetylphenyl)-4-methyl-1,3-thiazole-5-carboxylic acid (74.3 mg, 95%) as a white solid.

ESI-MS m/z 262 (M+H)⁺.

b)

According to the experimental procedure used in example I, the reaction between 2-(3-acetylphenyl)-4-methyl-1,3-thiazole-5-carboxylic acid (74.3 mg, 0.28 mmol) and methyl [(pyridin-2-ylmethyl)amino]acetate (61.5 mg, 0.34 mmol, prepared as described above) afforded methyl [{[2-(3-acetylphenyl)-4-methyl-1,3-thiazol-5-yl]carbonyl}(pyridin-2-ylmethyl)amino]acetate (60.5 mg, 50%) as an oil.

ESI-MS m/z 424 (M+H)⁺.

c)

According to the experimental procedure used in example I, saponification of methyl [{[2-(3-acetylphenyl)-4-methyl-1,3-thiazol-5-yl]carbonyl}(pyridin-2-ylmethyl)amino]acetate (60.5 mg, 0.14 mmol) led to [{[2-(3-acetylphenyl)-4-methyl-1,3-thiazol-5-yl]carbonyl}(pyridin-2-ylmethyl)amino]acetic acid (9.2 mg, 16%).

ESI-MS m/z 410 (M+H)⁺.

¹H NMR (CD₃OD) 2 rotamers in a 1/1 ratio, each chemical shift is for both rotamers except when stated, δ (ppm): 8.46-8.34 (m, 2H), 8.08-7.98 (m, 2H), 7.83-7.71 (m, 1H), 7.55-7.45 and 7.33-7.26 (m, 3H), 4.84-4.80 (m, 4H), 4.20-4.16 (m, 4H), 2.57 (s, 3H, one rotamer), 2.55 (s, 3H, one rotamer), 2.40 (br s, 3H).

### EXAMPLE XXXVII

### [{[2-(4-amino-3-nitrophenyl)-4-methyl-1,3-thiazol-5-yl]carbonyl}(pyridin-2-ylmethyl)amino]acetic Acid

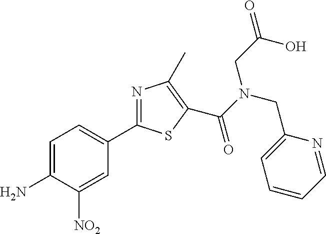

a)

Under argon, to a solution of 2-bromo-4-methyl-1,3-thiazole-5-carboxylic acid (229 mg, 1 mmol) in anhydrous dichloromethane (5 mL) at 0° C., were successively added a solution of oxalyl chloride (2M solution in dichloromethane, 0.6 mL, 1.2 mmol) and N,N-dimethylformamide (1 drop). The reaction mixture was stirred for 2.5 h allowing the temperature to rise to room temperature. Then a solution of methyl [(pyridin-2-ylmethyl)amino]acetate (180 mg, 1 mmol, prepared as in example I) in dichloromethane (5 mL) was added followed by N,N-diisopropylethylamine (0.61 mL, 3.5 mmol). The resulting mixture was stirred overnight. Water was added and the reaction mixture was extracted with dichloromethane. The combined organic extracts were dried over sodium sulfate, filtered and evaporated. The crude product was purified by flash chromatography (silica gel, dichloromethane/methanol 1/0 to 95/5) to afford a mixture of methyl [[(2-chloro-4-methyl-1,3-thiazol-5-yl)carbonyl](pyridin-2-ylmethyl)amino]acetate and methyl [[(2-bromo-4-methyl-1,3-thiazol-5-yl)carbonyl](pyridin-2-ylmethyl)amino]acetate (259 mg) as a brown oil.

ESI-MS m/z 340, 342, 384 and 386 (M+H)⁺.

b)

Under argon, a solution of methyl [[(2-halogeno-4-methyl-1,3-thiazol-5-yl)carbonyl](pyridin-2-ylmethyl)amino]acetate (159 mg of the mixture of 2-chloro and 2-bromo compounds obtained above), 2-nitro-4-(4,4,5,5-tetramethyl-1,3,2-dioxaborolan-2-yl)aniline (234 mg, 0.86 mmol), cesium carbonate (280 mg, 0.86 mmol), and tetrakis(triphenylphosphine)palladium (17.4 mg, 0.015 mmol) in degassed 1,4-dioxane (4 mL) and methanol (0.2 mL) was stirred at 80° C. overnight. The reaction mixture was filtered through a bed of celite and rinsed with dichloromethane, methanol and ethyl acetate. The solvents were evaporated and the crude product was purified by flash chromatography (silica gel, dichloromethane/methanol 9/1) to give methyl [{[2-(4-amino-3-nitrophenyl)-4-methyl-1,3-thiazol-5-yl]carbonyl}(pyridin-2-ylmethyl)amino]acetate (100 mg). The latter compound was dissolved in tetrahydrofuran (1 mL) and water (1 mL), lithium hydroxide (100 mg, 4.1 mmol) was added and the resulting mixture was stirred at room temperature overnight. An aqueous hydrochloric solution (1N) was added and the reaction mixture was extracted with diethyl ether, ethyl acetate, and dichloromethane. The combined organic extracts were dried over sodium sulfate, filtered and evaporated. Crystallization in a mixture of ethyl acetate, cyclohexane, dichloromethane and methanol afforded [{[2-(4-amino-3-nitrophenyl)-4-methyl-1,3-thiazol-5-yl]carbonyl}(pyridin-2-ylmethyl)amino]acetic acid (40 mg, 15% from 2-bromo-4-methyl-1,3-thiazole-5-carboxylic acid) as a red solid.

ESI-MS m/z 428 (M+H)⁺.

¹H NMR (CD₃OD), δ (ppm): 8.58-8.53 (m, 2H), 7.86-7.82 (m, 2H), 7.40-7.35 (m, 2H), 7.02 (d, J=8.2 Hz, 1H), 4.29-4.24 (m, 2H), 2.44 (s, 3H).

### EXAMPLE XXXVIII

### ((1,3-benzothiazol-2-ylmethyl){[2-(1H-indol-5-yl)-4-methyl-1,3-thiazol-5-yl]carbonyl}amino)acetic Acid

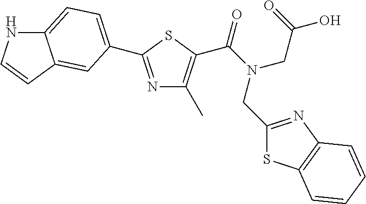

a)

Under argon, to a solution of 2-bromo-4-methyl-1,3-thiazole-5-carboxylic acid (80.5 mg, 0.36 mmol) in anhydrous dichloromethane (2 mL) at 0° C., were successively added a solution of oxalyl bromide (2M solution in dichloromethane, 190 μL, 0.38 mmol) and N,N-dimethylformamide (1 drop). The reaction mixture was stirred for 2 h allowing the temperature to rise to room temperature. Then at 0° C., a solution of ethyl [(1,3-benzothiazol-2-ylmethyl)amino]acetate (86.9 mg, 0.35 mmol, prepared as in example XXIX) in dichloromethane (1 mL) was added followed by N,N-diisopropylethylamine (0.2 mL, 1.1 mmol). The resulting mixture was stirred overnight allowing the temperature to rise to room temperature. Water was added and the reaction mixture was extracted with dichloromethane. The combined organic extracts were dried over sodium sulfate, filtered and evaporated. The crude product was purified by flash chromatography (silica gel, cyclohexane/ethyl acetate 1/0 to 7/3) to afford ethyl {(1,3-benzothiazol-2-ylmethyl)[(2-bromo-4-methyl-1,3-thiazol-5-yl)carbonyl]amino}acetate (124.5 mg, 79%) as a yellow oil.

ESI-MS m/z 454 and 456 (M+H)⁺.

b)

Under argon, a solution of {(1,3-benzothiazol-2-ylmethyl)[(2-bromo-4-methyl-1,3-thiazol-5-yl)carbonyl]amino}acetate (35.2 mg, 0.077 mmol), tert-butyl 5-(4,4,5,5-tetramethyl-1,3,2-dioxaborolan-2-yl)-1H-indole-1-carboxylate (35.6 mg, 0.10 mmol), cesium carbonate (50.5 mg, 0.15 mmol), and [1,1′-bis(diphenylphosphino)ferrocene]dichloropalladium(II) with dichloromethane (3.2 mg, 0.004 mmol) in degassed 1,4-dioxane (0.5 mL) and water (0.15 mL) was stirred at 110° C. for 2 days. The reaction mixture was filtered through a bed of celite and rinsed with dichloromethane, methanol and ethyl acetate. The solvents were evaporated and the crude product was purified by preparative TLC (silica gel, dichloromethane/methanol 9/1) to give ((1,3-benzothiazol-2-ylmethyl){[2-(1H-indol-5-yl)-4-methyl-1,3-thiazol-5-yl]carbonyl}amino)acetic acid (9.6 mg, 27%) as a beige solid.

ESI-MS m/z 463 (M+H)⁺.

¹H NMR (CD₃OD), δ (ppm): 8.23-8.13 (m, 1H), 8.04 (t, J=7.2 Hz, 2H), 7.75-7.65 (m, 1H), 7.85 (t, J=7.4 Hz, 1H), 7.50 (t, J=7.6 Hz, 2H), 7.37 (d, J=2.4 Hz, 1H), 6.60 (br s, 1H), 5.26-5.18 (m, 2H), 2.58 (br s, 3H).

The following compounds were prepared on a similar way:

### EXAMPLE XXXIX

### ((1,3-benzothiazol-2-ylmethyl){[4-methyl-2-(3-nitrophenyl)-1,3-thiazol-5-yl]carbonyl}amino)acetic Acid

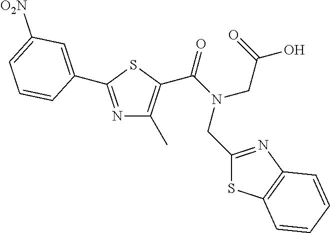

¹H NMR (CD₃OD), δ (ppm): 8.85-8.75 (m, 1H), 8.41-8.26 (m, 2H), 8.05 (t, J=8.6 Hz, 2H), 7.82-7.75 (m, 1H), 7.59 (t, J=7.4 Hz, 1H), 7.51 (t, J=7.6 Hz, 1H), 5.27-5.20 (m, 2H), 2.63 (br s, 3H).

### EXAMPLE XL

### ((1,3-benzothiazol-2-ylmethyl){[4-methyl-2-(2,6-dimethylphenyl)-1,3-thiazol-5-yl]carbonyl}amino)acetic Acid

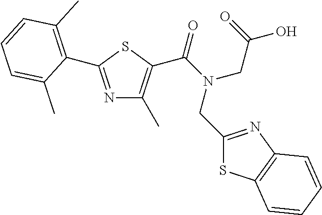

¹H NMR (CD₃OD) 2 rotamers in a roughly 1/2 ratio, each chemical shift is for both rotamers except when stated, δ (ppm): 8.06-8.02 (m, 2H), 7.58 (t, J=7.8 Hz, 1H), 7.50 (t, J=7.8 Hz, 1H), 7.34-7.29 (m, 3H, minor rotamer), 7.22-7.15 (m, 3H, major rotamer), 5.29-5.25 (m, 3H), 4.47 (br s, 2H, minor rotamer), 4.24 (br s, 2H, major rotamer), 2.62 (s, 3H), 2.22 (s, 6H, major rotamer), 2.11 (s, 6H, minor rotamer).

### EXAMPLE XLI

### ((1,3-benzothiazol-2-ylmethyl){[4-methyl-2-(2-naphthyl)-1,3-thiazol-5-yl]carbonyl}amino)acetic Acid

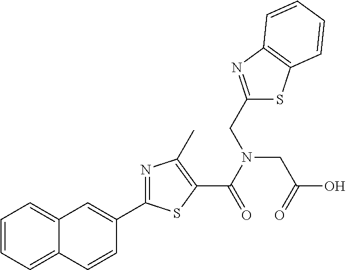

¹H NMR (DMSO-d₆), δ (ppm): 8.53-8.47 (m, 1H), 8.11-7.96 (m, 6H), 7.59 (br s, 2H), 7.52 (t, J=7.6 Hz, 1H), 7.45 (t, J=7.6 Hz, 1H), 5.13 (s, 2H), 4.30 (s, 2H).

### EXAMPLE XLII

### ((1,3-benzothiazol-2-ylmethyl){[4-methyl-2-(3-nitrophenyl)-1,3-thiazol-5-yl]carbonyl}amino)acetic Acid

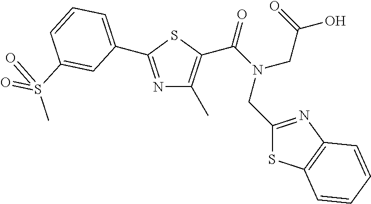

¹H NMR (CDCl₃), δ (ppm): 8.44-8.36 (m, 1H), 8.06-7.75 (m, 4H), 7.60-7-30 (m, 3H), 5.25-5.05 (m, 2H), 4.35-4.15 (m, 2H), 3.09 (br s, 3H), 2.57 (br s, 3H).

### EXAMPLE XLIII

### Inhibition of the Enzymatic Activity of RfaE

The IC50 values in μM are given in Table 1 hereinafter.

| | | IC50 |
|---|---|---|
| Example | | RfaE |
| N° | CHEMISTRY | (μM) |
| I | 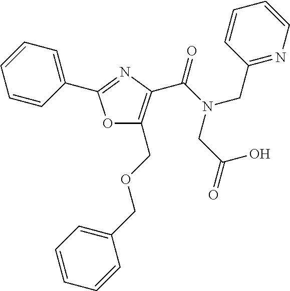 | 265 |
| XV | 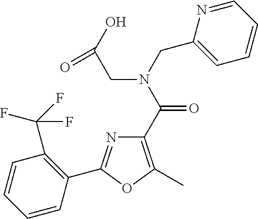 | 121 |
| XXXI | 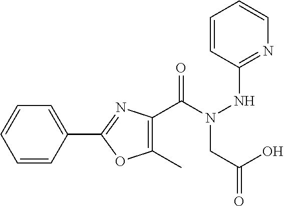 | 65 |
| XXXVI | 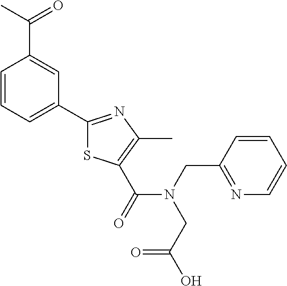 | 216 |
| XXXIII | 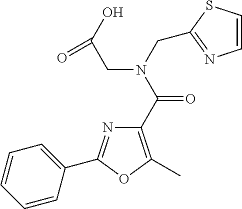 | 110 |
| VIII | 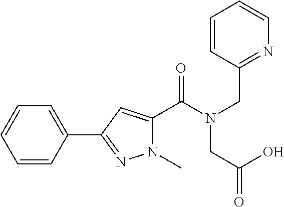 | 57 |
| II | 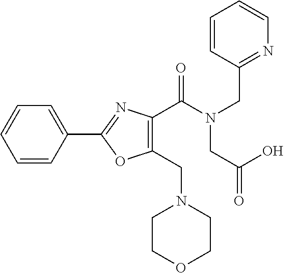 | 197 |
| VII | 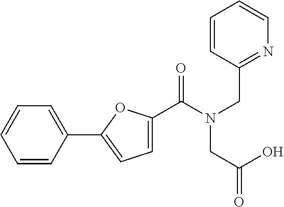 | 100 |
| V | 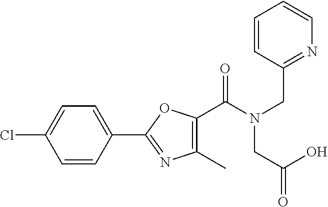 | 54 |
| XVI | 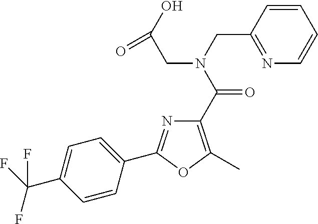 | 142 |
| XIII | 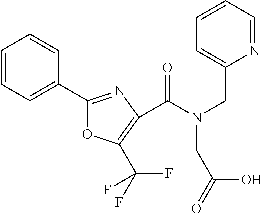 | 80 |
| IV | 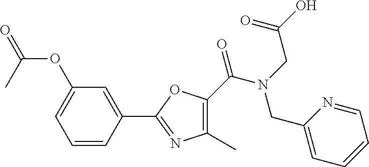 | 52 |
| XXII | 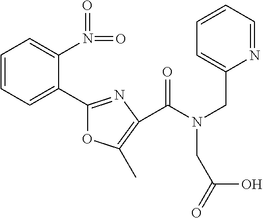 | 141 |
| XXI | 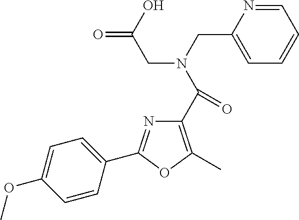 | 75 |
| IX | 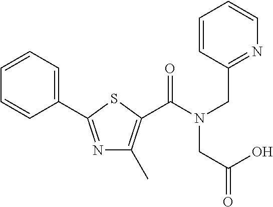 | 52 |
| XXXII | 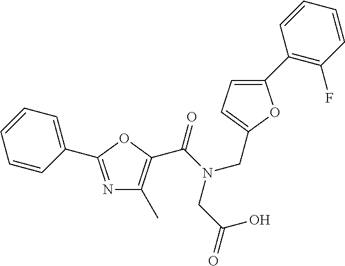 | 125 |
| VI | 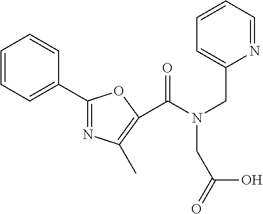 | 71 |
| XII | 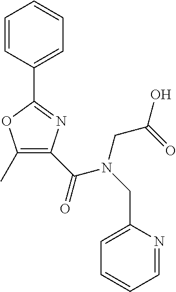 | 51 |
| XXV | 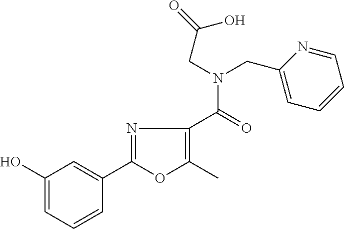 | 50 |
| XXVI | 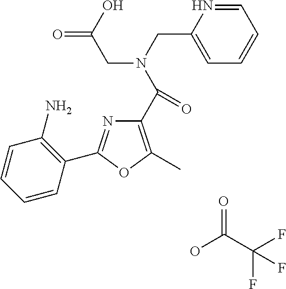 | 31 |
| XXXIV | 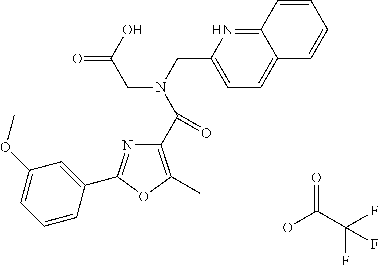 | 12 |
| XI | 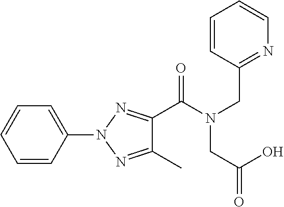 | 48 |
| XVII | 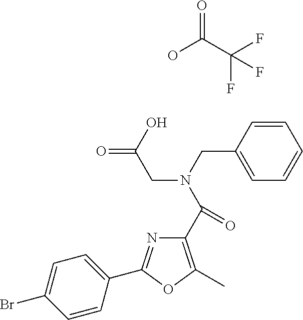 | 27 |
| XVIII | 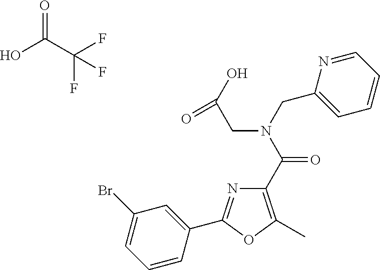 | 10 |
| XIX | 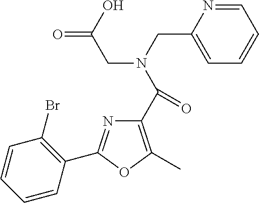 | 44 |
| XXVII | 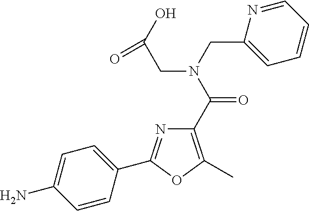 | 22 |
| XXIII | 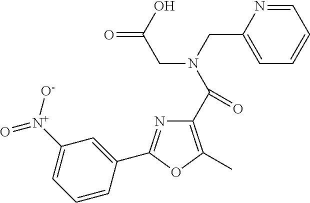 | 3.8 |
| X | 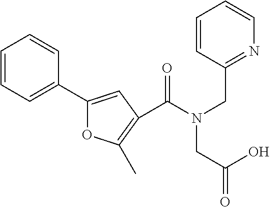 | 42 |
| III | 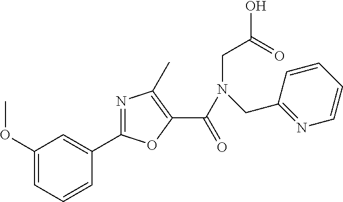 | 17 |
| XXXVIII | 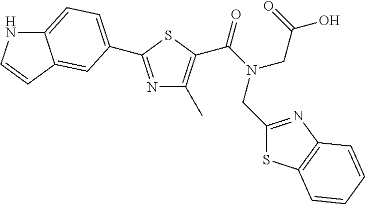 | 0.33 |
| XXIV | 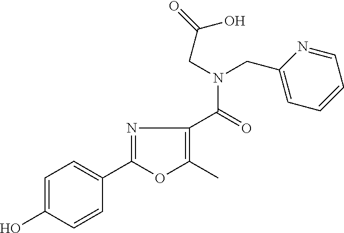 | 39 |
| XX | 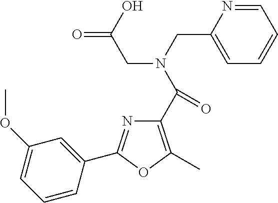 | 15 |
| XXXIX | 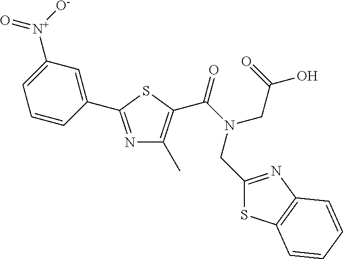 | 0.5 |
| XIV | 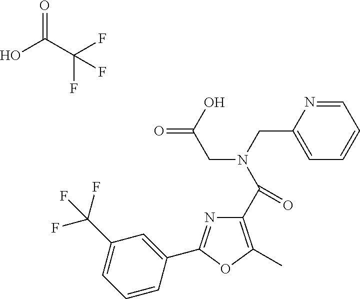 | 35 |
| XXVIII | 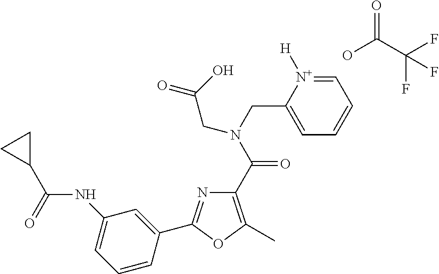 | 14 |
| XL | 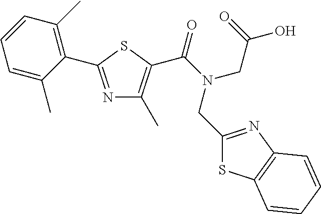 | 50.5 |
| XLI | 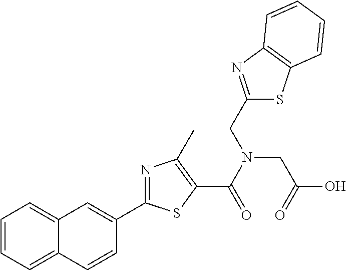 | 0.48 |
| XLII | 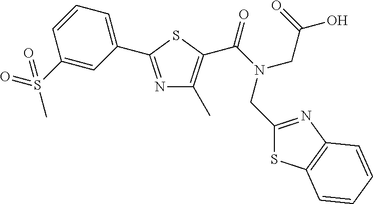 | 75 |
| XXXV | 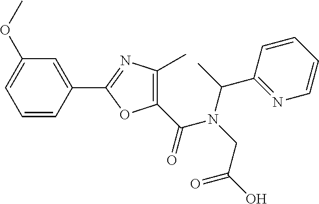 | 19.2 |
| XXIX | 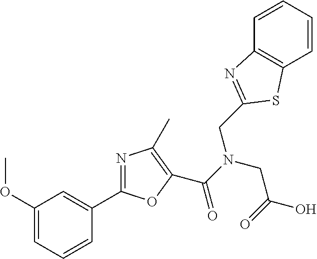 | 0.24 |
| XXX | 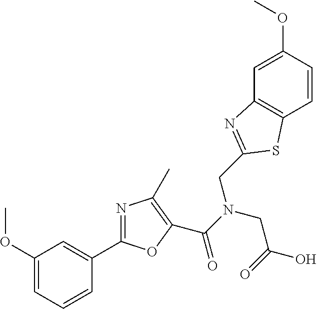 | 1.09 |
| XXXVII | 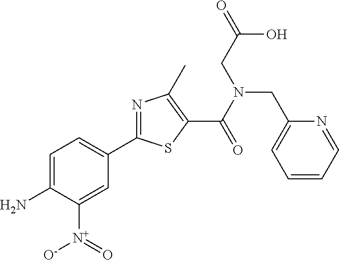 | 12.9 |

FIG. 1 illustrates the Dose Dependent Inhibition of RfaE Biochemical Activity by the Compound of Example XXIII

### EXAMPLE XLIV

### HTS Biochemical Assays Developed to Assess RfaE Enzymatic Activity

### Assays:

RfaE is a kinase belonging to the ribokinase family. It catalyses an essential step of the biosynthesis of L-ADP-Heptose, namely the phosphorylation of β-heptose-7-phosphate (H7P) into β-heptose-1,7-bisphosphate (H17P). RfaE assays as described in the literature are essentially based on direct HLPC detection of the substrates H7P and ATP, and of the products H17P and ADP, raising obvious limitations for HTS applications. The assays described below are based either on luminescent ATP detection, or on fluorescent ADP detection. They are easily amenable to miniaturized formats and fast readouts as required by HTS.

### RfaE Luminescent Assay

The assay buffer “AB” contains 50 mM Hepes pH7.5, 1 mM MnCl₂, 25 mM KCl, 0.012% Triton-X100 and 1 mM DTT. The following components are added in a white polystyrene Costar plate up to a final volume of 31 μL: 3 μL DMSO, or inhibitor dissolved in DMSO and 28 μL RfaE in AB. After 30 min of pre-incubation at room temperature, 29 μL of Substrates mix in AB are added in each well to a final volume of 60 μL. This reaction mixture is then composed of 3 nM RfaE (produced in house from E. coli), 0.2 μM β-heptose-7-phosphate (in house synthesis) and 0.2 μM ATP (Sigma) in assay buffer. After 40 min of incubation at room temperature, 200 μL of the revelation mix are added to a final volume of 260 μL, including the following constituents at the respective final concentrations: 2 nM luciferase (Sigma), 30 μM D-luciferin (Sigma), 100 μM N-acetylcysteamine (Aldrich). Luminescence intensity is immediately measured on an Analyst-HT (Molecular Devices) and converted into inhibition percentages. For IC50 determinations, the inhibitor is tested at 6 to 10 different concentrations, and the related inhibitions are fitted to a classical langmuir equilibrium model using XLFIT (IDBS).

### RfaE Fluorescent Assay

The assay buffer “AB” contains 50 mM Hepes pH7.5, 1 mM MnCl₂, 25 mM KCl, 0.012% Triton-X100 and 1 mM DTT. The following components are added in a black polystyrene Costar plate up to a final volume of 50 μL: 5 μL DMSO, or inhibitor dissolved in DMSO and 45 μL RfaE in AB. After 30 min of pre-incubation at room temperature, 50 μL of Substrates-revelation mix in AB are added in each well to a final volume of 100 μL. This reaction mixture is then composed of 66 μM RfaE (produced in house from E. coli), 1 μM β-heptose-7-phosphate (in house synthesis), 50 μM ATP (Sigma), 5 u/mL Pyruvate Kinase (Sigma), 50 μM phosphoenolpyruvate (Sigma), 5 u/mL Lactate dehydrogenase (Sigma) and 2.5 μM NADH (Sigma) in assay buffer. Fluorescence intensity of NADH (λₑₓ=360 nm, λₑₘ=520 nm) is immediately measured kinetically by a Fluostar Optima (BMG). Inhibition percentages are derived from fitted initial velocities. For IC50 determinations, the inhibitor is tested at 6 to 10 different concentrations, and the related inhibitions are fitted to a classical langmuir equilibrium model using XLFIT (IDBS).

1. Compounds having heptose synthesis inhibitory properties, of formula I 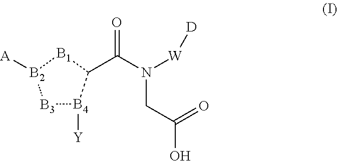
or a pharmaceutically acceptable salt, or prodrug thereof, wherein
A is an aryl or heterocycle, optionally substituted by one or several identical or different R such as H, C1-C10 alkyl, C1-C10 alkyl-OR₁, C1-C10 alkyl-NR₁R₁, alkoxy, hydroxy, thioalkyl, aryl, heterocycle, halogen, nitro, cyano, CO₂R₁, NR₁R₁, NR₁C(O)R₁, C(O)NR₁R₁, NR₁C(S)R₁, C(S)NR₁R₁, SO₂NR₁R₁, SO₂R₁, NR₁SO₂R₁, NR₁C(O)NR₁R₁, NR₁C(O)OR₁, NR₁C(S)NR₁R₁, NR₁C(S)OR₁, R₁C═NOR₁, C(O)R₁, aryloxy, thioaryl, alkenyl, alkynyl
R1 identical or different is H or C1-C10 alkyl
B₁, B₂, B₃ identical or not represent C, N, O, S to form a five-membered aromatic ring wherein from one to three carbon atoms are replaced by a heteroatom selected from S, O, N optionally substituted by one or several identical or different R such as defined above
B₄ is C or N
Y is H, C1-C10 alkyl, alkoxy, thio-alkyl, optionally substituted by one or several identical or different R such as defined above
W is C, O or N, substituted or not by one or several C1-C10 alkyl radicals
D is an heterocycle optionally substituted by one or several identical or different R such as defined above

2. The compounds of claim 1, wherein
A is an aryl or an heterocycle optionally substituted by one or several identical or different R such as defined in claim 1
B₁, B₂, B₃, identical or not represent C, N, O, S, to form a five-membered aromatic ring wherein from one to three carbon atoms are replaced by a heteroatom selected from S, O, N substituted or not by a C1-C10 alkyl
B₄ is C or N
Y is H or C1-C10 alkyl optionally substituted by one or several identical or different R such as defined above
W is C or N substituted or not by one or several C1-C10 alkyl radicals
D is a thiazole, benzothiazole, pyridine, or quinoline optionally substituted by one or several identical or different R such as defined in claim 1.

3. The derivatives of claim 2 wherein A is an aryl optionally substituted by one or several identical or different R.

4. The derivatives of claim 2 wherein A is an heterocycle optionally substituted by one or several identical or different R.

5. The derivatives of claim 1 wherein Y is a methyl or trifluoromethyl.

6. The derivatives of claim 1 wherein D is a 2-thiazole, 2-benzothiazole, 2-pyridine, or 2-quinoline optionally substituted by one or several identical or different R.

7. The compounds according to claim 1 under the racemic forms or the enantiomers thereof.

8. The tautomeric forms of compounds according to claim 1.

9. The salts of compounds according to claim 1.

10. A method for the synthesis of compounds according to claim 1 comprising
a—reacting compounds of formula II or their salt forms: 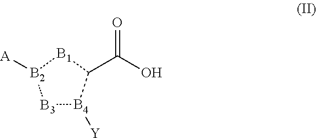
wherein A, B₁, B₂, B₃, B₄ and Y are as above defined; with a compound of formula III or its salt form: 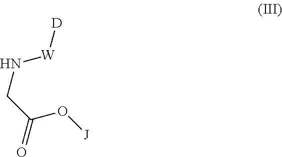
wherein D and W are as above defined, J is a C1-C10 alkyl group optionally substituted by one or several identical or different R such as defined above, under conditions resulting in the formation of an amide bond;
b—reacting compounds of formula IV or their salt forms: 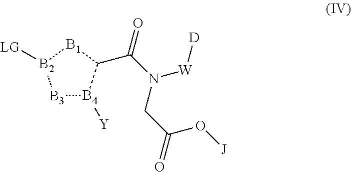
wherein B₁, B₂, B₃, B₄, D, W and Y are as above defined, LG is a leaving group such as a halogen or a sulfonyloxy group. J is a C1-C10 alkyl group optionally substituted by one or several identical or different R such as defined above; with a compound of formula V, or its salt form: 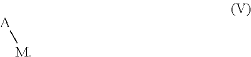
wherein A is as above defined, M represents H, B(OH)₂, B(OR)₂, BF₃K, or any metal atom substituted or not by R groups different or not, with R as above defined,
c—reacting compounds of formula VI, or their salt forms: 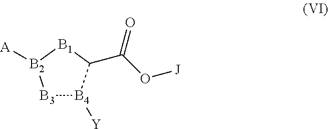
wherein A, B₁, B₂, B₃, B₄, Y are as above defined, J is a C1-C10 alkyl group optionally substituted by one or several identical or different R such as defined above; with a compound of formula II, or a salt thereof as above described.
d—Transforming compounds into other compounds by a reaction of the group comprising deprotection, alkylation, acylation, nucleophilic substitution, reduction, oxidation, transition metal catalyzed reaction.

11. The method of claim 10 wherein the ester obtained according to step a or b or step c is converted into the corresponding carboxylic acid by hydrolysis or saponification.

12. The method of claim 10, wherein
the compounds of formula II and their salt forms are obtained by saponification or hydrolysis of an ester, or by a deprotection reaction of protected acid functionalities of compounds of formula VI or their salt forms.
the compounds of formula VI and their salt forms are synthesized by reaction of compounds of formula VII or their salt forms: 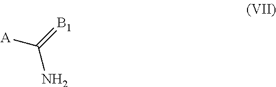
wherein A is as above defined and is O or S; with a compound of formula VIII or its salt form: 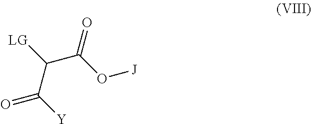
wherein Y is as above defined, LG is a leaving group such as a halogen or a sulfonyloxy group, J is a C1-C10 alkyl group optionally substituted by one or several identical or different R such as defined above, or alternatively
the compounds of formula VI and their salt forms are synthesized by reaction of compounds of formula IX, or their salt forms: 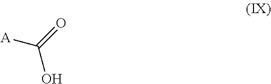
wherein A is as above defined; with a compound of formula X or its salt form: 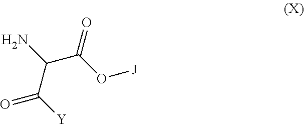
wherein Y is as above defined, J is a C1-C10 alkyl group optionally substituted by one or several identical or different R such as defined above
or alternatively
the compounds of formula VI, and their salt forms, are prepared by the reaction of compounds of formula VII or their salt forms as above defined, with a compound of formula XI or its salt form: 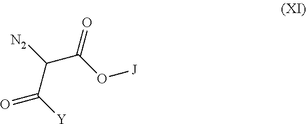
wherein Y is as above defined, J is a C1-C10 alkyl group optionally substituted by one or several identical or different R such as defined above,
or alternatively
the compounds of formula VI and their salt forms are prepared by the reaction of compounds of formula XII or their salt forms: 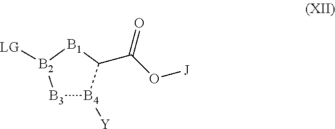
wherein B₁, B₂, B₃, B₄, and Y are as above defined; LG is a leaving group such as a halogen or a sulfonyloxy group, J is a C1-C10 alkyl group optionally substituted by one or several identical or different R such as defined above, under nucleophilic substitution or metal-mediated coupling conditions to displace the leaving group LG
with a compound of formula V, or its salt form,
Optionally, the compounds of formula VI and their salt forms are further chemically modified by using a reaction selected in the group comprising deprotection, alkylation, acylation, nucleophilic substitution, reduction, oxidation, transition metal catalyzed reaction to provide other compounds of formula VI and their salt forms
the compounds of formula II and their salt forms are prepared by reaction of a compound of formula XIII or a salt or its salt form: 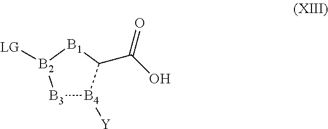
wherein B₁, B₂, B₃, B₄ and Y are as above defined, LG is a leaving group such as a halogen or a sulfonyloxy group,
with a compound of formula V, or its salt form as above defined by nucleophilic substitution or metal-mediated coupling reaction,
Optionally, the compounds of formula II and their salt forms are further chemically modified by using a reaction selected in the group comprising deprotection, alkylation, acylation, nucleophilic substitution, reduction, oxidation, transition metal catalyzed reaction to provide other compounds of formula II and their salt forms
the compounds of formula III and their salt forms are prepared by reaction of a compound of formula XIV, or its salt form: 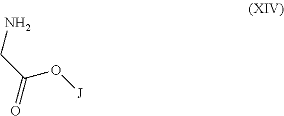
wherein J is a C1-C10 alkyl group optionally substituted by one or several identical or different R such as defined above; with a compound of formula XV, or its salt form: 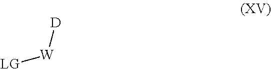
wherein D and W are as above defined and LG is a leaving group such as a halogen or a sulfonyloxy group or alternatively
the compounds of formula III and their salt forms are prepared by reaction of a compound of formula XVI, or its salt form: 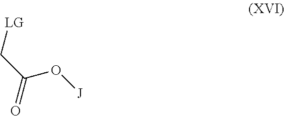
wherein LG is a leaving group such as a halogen or a sulfonyloxy group, J is a C1-C10 alkyl group optionally substituted by one or several identical or different R such as defined above; with a compound of formula XVII, or its salt form: 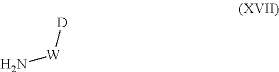
wherein D and W are as above defined, under nucleophilic substitution conditions, or alternatively
the compounds of formula III and their salt forms are prepared by reaction of a compound of formula XVIII, or its salt form: 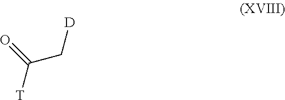
wherein D is as above defined and T is H or C1-C10 alkyl as defined herein previously;
with a compound of formula XIV or its salt form as above defined, under reductive amination conditions, or alternatively
the compounds of formula III and their salt forms are synthesized by reaction of a compound of formula XIX, or its salt form: 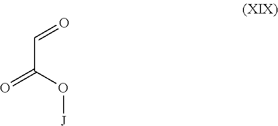
wherein J is a C1-C10 alkyl group optionally substituted by one or several identical or different R such as defined above; with a compound of formula XVII, or its salt form, as above defined, under reductive amination conditions
Optionally, the compounds of formula III and their salt forms are further chemically modified by using a reaction selected in the group comprising deprotection, alkylation, acylation, nucleophilic substitution, reduction, oxidation, transition metal catalyzed reaction to provide other compounds of formula III and their salt forms
the compounds of formula IV and their salt forms are prepared by reaction of a compound of formula XIII or its salt form with a compound of formula III or its salt form, as defined herein previously.

13. The derivatives of claim 1, further characterized by the following properties: they are able to inhibit the activity of RfaE enzyme

14. A method for assessing RfaE enzymatic activity
a. pre-incubating at room temperature
DMSO or inhibitor to be tested dissolved in DMSO and RfaE in an assay buffer
and either
adding a reaction mixture composed of RfaE, β-heptose-7-phosphate, ATP, in the assay buffer and incubating at room temperature
adding a revelation mixture composed of luciferase, D-luciferin and N-acetylcysteamine
measuring the luminescence intensity and converting into inhibition % to further calculate the IC₅₀ values;
or
adding a reaction mixture composed of RfaE, β-heptose-7-phosphate ATP, pyruvate kinase, phosphoenolpyruvate, lactate dehydrogenase and NADH in said assay buffer,
measuring the fluorescence intensity of NADH kinetically and deriving inhibition % from fitted initial velocities, to further calculate the IC₅₀ values.

15. A composition comprising at least a derivative of formula (I) such as defined in claim 1, for use as drug.

16. The composition of claim 15 for use as antibacterial agent to treat Gram-negative bacterial infections in human and animals, particularly to treat infections due to following Gram negative species (spp): Escherichia coli, Enterobacter, Salmonella, Shigella, Pseudomonas, Acinetobacter, Neisseria, Klebsiella, Serratia, Citrobacter, Proteus, Yersinia, Haemophilus, Legionella, Moraxella and Helicobacter pylori.

17. A pharmaceutical composition comprising an effective amount of at least one derivative of formula (I) such as defined in claim 1 in combination with a pharmaceutically acceptable carrier.

18. A pharmaceutical composition comprising an effective amount of at least one derivative of formula (I) such as defined in claim 1, in combination with an antibacterial molecule and a pharmaceutically acceptable carrier.

19. The pharmaceutical composition according to claim 16, which is formulated to be administered under oral, injectable, parenteral routes, with individual doses appropriate for the patient to be treated.
